(19) United States
(12) Patent Application Publication
(10) Pub. No.: US 2005/0119262 A1
(43) Pub. Date: Jun. 2, 2005

(54) Method for preventing or treating an optic neuropathy with a cox-2 inhibitor and an intraocular pressure reducing agent

(75) Inventor: Martin B. Wax, Westlake, TX (US)

Correspondence Address: James E. Davis;Harness, Dickey & Pierce, P.L.C., 7700 Bonhomme, Suite 400, Clayton, MO, 63105, US

(73) Assignee: Pharmacia Corporation, Chesterfield, MO (US)

(21) Appl. No.: 10/919,835

(22) Filed: Aug. 17, 2004

Related U.S. Application Data
(60) Provisional application No. 60/497,043, filed on Aug. 21, 2003.

(51) Int. Cl. A61K 31/5377; A61K 31/415; A61K 31/557; A61K 31/137

(52) U.S. Cl. 514/235.5; 514/406; 514/573; 514/469; 514/411; 514/649; 514/738; 514/509

(57) ABSTRACT

The present invention provides methods and compositions for the prevention and/or treatment of an optic neuropathy, comprising a Cox-2 inhibitor and an intraocular pressure reducing agent.

### CROSS-REFERENCE TO RELATED PATENTS AND PATENT APPLICATIONS

This application is a non-provisional of and claims priority to U.S. Provisional Patent Application Ser. No. 60/497,043 filed Aug. 21, 2003, which is incorporated by reference herein in its entirety.

### BACKGROUND OF THE INVENTION

(1) Field of the Invention

The present invention relates to a method for preventing or treating an optic neuropathy, and more particularly to a method for preventing or treating an optic neuropathy with a Cox-2 inhibitor in combination with an intraocular pressure reducing agent in a subject that is in need of such prevention or treatment, and to compositions and kits that are useful for effecting the method.

(2) Description of the Related Art

An important segment of ophthalmologic disorders involve diseases, disorders, or injury to nerves associated with the eye. These optic neuropathies include glaucoma, ocular hypertension, compressive and infiltrative neuropathies, retinopathies, tumors of the optic nerve, and inflammatory, toxic, traumatic, vascular and hereditary optic nerve diseases, as well as others.

Important among these disorders is glaucoma, which is the second most common cause of blindness in the USA. Glaucoma is a group of diseases which are generally characterized by elevated intraocular pressure that damages the optic nerve. See, e.g., Gittinger, J. W., Jr., Eye Diseases, pp. 2269-2279, in Cecil Textbook of Medicine, 19^{th} Ed., J. B. Wyngaarden, L. H. Smith, and J. C. Bennett, Eds., W. B. Saunders Co., Philadelphia, Pa. (1992). Currently, treatment of glaucoma is primarily medical and includes administration of topical parasympathomimetics, (e.g., pilocarpine and carbachol), beta-andrenergic blockers (e.g., timolol, betzolol, and levobunolol), sympathomimetics (e.g., echothiophate), and, more recently, agents which lower intraocular pressure, such as latanoprost (XALATAN®), or systemic carbonic anhydrase inhibitors (e.g., acetazolamide and methazolamide). If medical therapy fails to halt progression of the disease, surgery is indicated, but often does not offer permanent relief.

Despite recent advances, vision preservation remains a key issue for the treatment of glaucoma. Therefore, there is ongoing research in improving existing treatment regimens as well as developing alternative treatment options.

Recently, several studies utilizing non-steroidal anti-inflammatory drugs (NSAIDs), either topically (see Kasiwagi, K. and Tsukahara, S., Br J Ophthalmol, 87:297-301 (2003); Baudouin, C., et al., Graefes Arch Clin Exp Ophthalmol, 240:929-35 (2002)) or orally (see Sponsel, W. E., et al., Am J Ophthalmol, 133:11-18 (2002)), in combination with conventional IOP-reducing agents have shown some promise. In contrast, the administration of the NSAID acetylsalicylic acid (aspirin) has been shown to have no affect on IOP in patients with ocular hypertension and glaucoma. See Linden, C. and Alm, A., Exp Eye Res, 70:281-3 (2000).

It is now recognized that many of the traditional NSAIDs are inhibitors of two cyclooxygenases, cyclooxygenase-1 (Cox-1) and cyclooxygenase-2 (Cox-2). These two enzymes are involved in the critical initiation step of prostaglandin synthesis; the addition of molecular oxygen to arachidonic acid in the cell membrane. See Needleman, P. et al., Annu Rev Biochem, 55:69-102 (1986).

Cox-1 is constituitively active and is responsible for the synthesis of housekeeping prostaglandins critical to maintaining normal renal function, gastric mucosal integrity, and vascular homeostasis. Cox-2 expression is induced by cytokines and growth factors in inflammatory cells, leading to the release of prostanoids (prostaglandin E2) which sensitize peripheral nociceptor terminals and produce localized pain hypersensitivity, inflammation, and edema. See e.g. Samad, T. A. et al., Nature 410:471-5 (2001). Because many common NSAIDs inhibit prostaglandin synthesis by blocking the activity of both Cox-1 and Cox-2, side effects associated with long-term administration of these drugs such as gastrointestinal bleeding and ulcers are thought to be a result of inhibiting the homeostatic functions of Cox-1, while the inhibiton of Cox-2 accounts for their analgesic properties.

Research into the area of arachidonic acid metabolism has resulted in the discovery of compounds that inhibit the cyclooxygenase-2 enzyme to a greater extent than the activity of Cox-1. The Cox-2-selective inhibitors are believed to offer advantages that include the capacity to prevent or reduce inflammation while avoiding harmful side effects associated with the inhibition of Cox-1. Thus, cyclooxygenase-2 selective inhibitors have shown great promise for use in therapies, especially in therapies that require maintenance administration.

While the effects of Cox-2 inhibitors on inflammation and inflammation-related disorders have been relatively widely recognized, it is not known whether the inhibition of Cox-2 would be an effective therapy for optic neuropathies, or whether the delivery of a Cox-2 inhibitor, across the sclera, could be accomplished sufficiently to provide a useful therapeutic method that did not depend upon intravitreal administration.

Accordingly, it would be useful to provide a method and compositions for the treatment of optic neuropathies, and in particular, for glaucoma. It would also be useful if the method and compositions were efficacious, safe, and easy to administer.

### SUMMARY OF THE INVENTION

Briefly, therefore the present invention is directed to a novel method for the prevention or treatment of an optic neuropathy in a subject, the treatment comprising administering to the subject a Cox-2 inhibitor and an intraocular pressure reducing agent. The method is particularly useful when the subject is one that is in need of prevention or treatment of an optic neuropathy.

The present invention is also directed to a novel composition comprising a Cox-2 inhibitor and an intraocular pressure reducing agent. The composition is useful for the prevention and/or treatment of an optin neuropathy.

The present invention is also directed to a novel pharmaceutical composition comprising a pharmaceutically-acceptable excipient and a combination comprising a Cox-2 inhibitor and an intraocular pressure reducing agent.

The present invention is also directed to a novel kit that is suitable for use in the prevention or treatment of an optic neuropathy, the kit comprising a first dosage form comprising a Cox-2 inhibitor and a second dosage form comprising an intraocular pressured reducing agent or prodrug thereof, in quantities which comprise a therapeutically effective amount of the combination of the compounds for the prevention or treatment of the optic neuropathy.

Among the several advantages found to be achieved by the present invention, therefore, may be noted the provision of a method and compositions for the treatment of optic neuropathies, and in particular, for glaucoma, and the provision of such method and compositions that are efficacious, safe, and easy to administer.

### DETAILED DESCRIPTION OF THE PREFERRED EMBODIMENTS

In accordance with the present invention, it has been discovered that an effective method for the prevention or treatment of an optic neuropathy in a subject comprises the administration to the subject of a Cox-2 inhibitor and an intraocular pressure reducing agent. In one embodiment of the present invention, the subject is one that is in need of such prevention or treatment.

In a preferred embodiment, the method comprises administering to a subject that is in need of such treatment an amount of a Cox-2 inhibitor, which, in combination with an amount of an intraocular pressure reducing agent, provides an amount of the combination that is effective for the treatment of the optic neuropathy. The effective amount of the combination is preferably a therapeutically effective amount.

The phrases “therapeutic amount”, “therapeutically-effective”, and “effective for the prevention or treatment” are intended to qualify the amount of each agent for use in the combination therapy which will achieve the goal of improvement in the severity of the optic neuropathy and the frequency of incidence over treatment of each agent by itself, while avoiding adverse side effects typically associated with alternative therapies.

The administration of a Cox-2 inhibitor in combination with an intraocular pressure reducing agent is an effective treatment for optic neuropathies and complications thereof, and in preferred embodiments, is superior to the use of either agent alone.

Moreover, in preferred embodiments, the combination therapies of the present invention demonstrate a synergistic efficacy for treating and preventing optic neuropathies that is greater than what would be expected from simply combining any of the individual monotherapies.

As used herein, the term “synergistic” refers to the combination of a Cox-2 inhibitor and an intraocular pressure reducing agent as a combined therapy having an efficacy for the prevention and treatment of optic neuropathies that is greater than what would be expected merely from the sum of their individual effects.

The synergistic effects of the embodiments of the present invention's combination therapies encompass additional unexpected advantages for the treatment and prevention of optic neuropathies. Such additional advantages include, but are not limited to, lowering the required dose of intraocular pressure reducing agents, reducing the side-effects of intraocular pressure reducing agents, and rendering those agents more tolerable to subjects in need of therapy for an optic neuropathy.

As used herein, the phrases “combination therapy”, “co-administration”, “co-administering”, “administration with”, “administering”, “combination”, or “co-therapy”, when referring to use of a Cox-2 inhibitor in combination with an intraocular pressure reducing agent, are intended to embrace administration of each agent in a sequential manner in a regimen that will provide beneficial effects of the drug combination, and is intended to embrace co-administration of these agents in a substantially simultaneous manner as well. Thus, the Cox-2 inhibitor and intraocular pressure reducing agent may be administered in one therapeutic dosage form, such as in a single capsule, tablet, eye drop, or injection, or in two separate therapeutic dosage forms, such as in separate capsules, tablets, eye drops, or injections.

Sequential administration of such treatments encompasses both relatively short and relatively long periods between the administration of each of the drugs of the present method. However, for purposes of the present invention, the second drug is administered while the first drug is still having an efficacious effect on the subject. Thus, the present invention, in one embodiment, takes advantage of the fact that the simultaneous presence of the combination of a Cox-2 inhibitor and an intraocular pressure reducing agent in a subject has a greater efficacy than the administration of either agent alone.

Preferably, the second of the two drugs is administered to the subject within the therapeutic response time of the first drug to be administered. For example, the present invention encompasses administration of a Cox-2 inhibitor to the subject and the later administration of an intraocular pressure reducing agent, as long as the intraocular pressure reducing agent is administered to the subject while the Cox-2 inhibitor is still present in the subject at a level, which in combination with the level of the intraocular pressure reducing agent is therapeutically effective, and vice versa. As used herein, the terms “therapeutic response time” mean the duration of time after administration that a compound has a therapeutic effect within a subject's body.

The terms “prevent”, “preventing”, or “prevention of” mean to preclude the development or occurrence of a disorder. In the context of this invention, that disorder is an optic neuropathy and, in particular, glaucoma.

As used herein, the terms “treating” or “to treat,” mean to alleviate symptoms, eliminate the causation either on a temporary or permanent basis, or to prevent or slow the appearance of symptoms. The term “treatment” includes alleviation, elimination of causation of, or prevention of symptoms. In the context of this invention, the symptoms are those associated with an optic neuropathy and, in particular, glaucoma.

The term “subject” for purposes of treatment includes any vertebrate. Preferably, the vertebrate is a human or animal subject who is in need of prevention or treatment for an optic neuropathy. The subject is typically a mammal. “Mammal”, as that term is used herein, refers to any animal classified as a mammal, including humans and non-human animals, such as domestic and farm animals, and zoo, sports, or pet animals, such as dogs, horses, cats, cattle, etc., Preferably, the mammal is a human. A subject “that is in need of the prevention or treatment”, is a subject who, by genetics, lifestyle, age, physical condition, accident, medical treatment, medical history, or otherwise, is at risk for contacting, or who has contacted, a disease or disorder. In the context of this application, the disease or disorder is an optic neuropathy.

The intraocular pressure reducing agent (IOP reducing agent) of the present invention can be any compound or combination of compounds that is capable of reducing intraocular pressure, no matter how slight the reduction. It is preferred that the IOP reducing agent reduce ocular pressure without causing damage to the eye. Examples of IOP reducing agents that are suitable for use in the present invention include direct-acting miotics, such as cholinergic agonists; indirect-acting miotics, such as cholinesterase inhibitors; carbonic anhydrase inhibitors; nonselective adrenergic agonists; α₂-selective adrenergic agonists; β-blockers; prostaglandins; prostaglandin analogues; osmotic diuretics; p38 kinase antagonists, salts thereof, isomer thereof, prodrugs thereof, and mixtures of any of these.

Examples of direct-acting miotics include pilocarpine, carbachol and acetylcholinesterase inhibitors. Examples of indirect-acting miotics include physostigmine, neostigmine, demecarium, echothiophate iodide, and isoflurophate. Examples of carbonic anhydrase inhibitors include acetazolamide, dichlorphenamide, methazolamide, ethoxzolamide, dorzolamide, and compounds disclosed in U.S. Pat. Nos. 5,153,192, 5,240,923, 5,378,703, and 4,797,413. Examples of nonselective adrenergic agonists include epinephrine, dipivalylepinephrine, para-amino clonidine, and dipivefrin. Examples of α₂-selective adrenergic agonists include aprachionidine and brimonidine. Examples of β-blockers include timolol, betaxolol, levobunolol, carteolol, and metipranolol. Examples of prostaglandins and prostaglandin analogues include F series (such as PGF_{2α}), E series (such as PGE₂), D series (such as PGD₂) and compounds disclosed in U.S. Pat. Nos. 4,132,847, 4,599,353, 5,093,329, 5,151,444, 5,173,507, 5,208,256, 5,262,437, 5,321,128, 5,462,968, 5,476,872, 5,565,492, 5,578,618, and 6,037,368 and in European Patent Nos. 0 215 860 B1 and 0 299 914 B1, and in WO 94/11002. Examples of osmotic diuretics include glycerin, mannitol, and isosorbide.

Examples of prostaglandin F_{2α} analogues that are useful in the present method include latanoprost, travoprost, AL-5848, PhXA85, and unoprostone.

Latanoprost has a chemical name of isopropyl-(Z)-7[(1R,2R,3R,5S)3,5-dihydroxy-2-[(3R)-3-hydroxy-5-phenylpentyl]cyclopentyl]-5-heptenoate; a chemical composition of C₂₆H₄₀O₅; and a molecular weight of 432.58. Latanoprost is available under the trade name Xalatan® from Pharmacia-Upjohn, Kalamazoo, Mich. The chemical structure of latanoprost, and a method for its production, are disclosed in WO 93/00329. The chemical structure of latanoprost is:

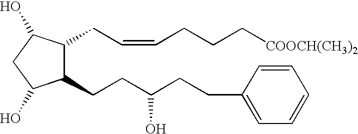

A novel process for making latanoprost is described in U.S. Pat. No. 5,466,833, and the use of latanoprost for treating glaucoma is described in U.S. Pat. No. 5,510,383.

Travoprost, having a chemical name of isopropyl(Z)-[(1R,2R,3R,5S)-3,5-dihydroxy-2-[(1E,3R)-3-hydroxy-4-[(α,α,α-trifluoro-m-tolyl)oxy]-1-butenyl]cyclopropentyl]-5-heptanoate, is available under the trade name of Travatan® from Alcon Pharmaceuticals, Alcon Laboratories, Inc., Fort Worth, Tex.

AL-5848 has a chemical name of (5Z,13E)-(9S,11R,15S)-9,11,15-trihydroxy-5,13-prostadienoic acid. It is the carboxylic acid form of travoprost, and a single (+)-isomer of (+/−)-fuprostenol, an FP-class prostaglandin agonist which lowers intraocular pressure.

Unoprostone is commonly available as its isopropyl ester, which has a chemical name of isopropyl(+)-(Z)-7-[(1R,2R,3R,5S)-3,5-dihydroxy-2-(3-ocodecyl)cyclopentyl]-5-heptanoate. Unoprostone is available commercially in the form of unoprostone isopropyl under the trade name Rescula®, from CIBA Vision, Duluth, Ga.

PhXA85 is lantanoprost acid, and is believed to be the biologically active form of latanoprost.

Other compounds, such as prostamide compounds, are sometimes classified along with prostaglandin analogues. However, for the purposes of the present specification, prostamide compounds, such as bimatoprost, will be considered to be included within the terms “prostaglandin analogue”.

The IOP reducing agent of the present invention can be supplied in any physical form, including liquids, gels, powders, crystals, or the like, and in any purity that is suitable for use in a pharmaceutical formulation. It is preferred that the IOP reducing agent be of U.S.P. degree of purity, or better. The IOP reducing agent can include small amounts of normal contaminants or by-products, so long as the contaminant or by-product does not interfere with the effectiveness of the present method, or cause a safety or stability problem in any formulation or composition that includes the novel combination.

One component of the present invention is a Cox-2 inhibitor.

Inhibitors of the Cox pathway in the metabolism of arachidonic acid may inhibit enzyme activity through a variety of mechanisms. By way of example, the Cox-2 inhibitors used in the methods described herein may block the enzyme activity directly by binding at the substrate site of the enzyme. In preferred embodiments, the use of a Cox-2 selective inhibitor is highly advantageous in that it minimizes the gastric side effects that can occur with non-selective non-steroidal anti-inflammatory drugs (NSAIDs), especially where prolonged treatment is expected.

The terms “cyclooxygenase-2 inhibitor”, or “Cox-2 inhibitor”, which can be used interchangeably herein, embrace compounds, which inhibit the Cox-2 enzyme regardless of the degree of inhibition of the Cox-1 enzyme, and include pharmaceutically acceptable salts of those compounds. Thus, for purposes of the present invention, a compound is considered a Cox-2 inhibitor irrespective of whether the compound inhibits the Cox-2 enzyme to an equal, greater, or lesser degree than the Cox-1 enzyme.

In one embodiment of the present invention, it is preferred that the Cox-2 inhibitor compound is a non-steroidal anti-inflammatory drug (NSAID). Therefore, preferred materials that can serve as the Cox-2 inhibitor of the present invention include non-steroidal anti-inflammatory drug compounds, a pharmaceutically acceptable salt thereof, mixed isomer, or a pure (−) or (+) optical isomeric form thereof.

Examples of NSAID compounds that are useful in the present invention include acemetacin, acetyl salicylic acid, alclofenac, alminoprofen, azapropazone, benorylate, benoxaprofen, bucloxic acid, carprofen, choline magnesium trisalicylate, clidanac, clopinac, dapsone, diclofenac, diflunisal, droxicam, etodolac, fenoprofen, fenbufen, fenclofenec, fentiazac, floctafenine, flufenisal, flurbiprofen, (r)-flurbiprofen, (s)-flurbiprofen, furofenac, feprazone, flufenamic acid, fluprofen, ibufenac, ibuprofen, indometacin, indomethacin, indoprofen, isoxepac, isoxicam, ketoprofen, ketorolac, miroprofen, piroxicam, meloxicam, mefenamic, mefenamic acid, meclofenamic acid, meclofen, nabumetone, naproxen, niflumic acid, nitroflurbiprofen, oxaprozin, oxipinac, oxyphenbutazone, phenylbutazone, podophyllotoxin derivatives, proglumetacin, piprofen, pirprofen, prapoprofen, salicylic acid, salicylate, sudoxicam, suprofen, sulindac, tenoxicam, tiaprofenic acid, tiopinac, tioxaprofen, tolfenamic acid, tolmetin, zidometacin, zomepirac, and 2-fluoro-a-methyl[1,1′-biphenyl]-4-acetic acid, a 4-(nitrooxy)butyl ester, and mixtures thereof.

Further preferred NSAID compounds include ibuprofen, naproxen, sulindac, ketoporfen, fenoprofen, tiaprofenic acid, suprofen, etodolac, carprofen, ketrolac, piprofen, indoprofen, salicylic acid, flurbiprofen, and mixtures thereof.

In a preferred embodiment, the Cox-2 inhibitor is a Cox-2 selective inhibitor. The term “Cox-2 selective inhibitor” embraces compounds, which selectively inhibit the Cox-2 enzyme over the Cox-1 enzyme, and also include pharmaceutically acceptable salts and prodrugs of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of Cox activity. Preferred Cox-2 selective inhibitors of the present invention have a Cox-2 IC₅₀ of less than about 1 μM, more preferred of less than about 0.5 μM, and even more preferred of less than about 0.2 μM.

Preferred Cox-2 selective inhibitors have a Cox-1 IC₅₀ of greater than about 1 μM, and more preferably of greater than 20 μM. Such preferred selectivity may indicate an ability to reduce the incidence of common NSAID-induced side effects.

Also included within the scope of the present invention are compounds that act as prodrugs of Cox-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic Cox-2 selective inhibitor valdecoxib. An example of a preferred Cox-2 selective inhibitor prodrug is sodium parecoxib. A class of prodrugs of Cox-2 inhibitors is described in U.S. Pat. No. 5,932,598.

In one embodiment of the present invention, the Cox-2 selective inhibitor is of the chromene/chroman (“chromene”) structural class, which encompasses substituted benzopyrans or substituted benzopyran analogs, as well as substituted benzothiopyrans, dihydroquinolines, or dihydronaphthalenes having the structure of general Formula I, shown below, and including, by way of non-limiting example, the chromene compounds described below, and the diastereomers, enantiomers, racemates, tautomers, salts, esters, amides and prodrugs thereof.

Chromenes that can serve as a Cox-2 selective inhibitor of the present invention include any one or more of the compounds that are described in U.S. Pat. Nos. 6,271,253; 6,492,390; 6,034,256 and 6,077,850. One such class of compounds is defined by the general formula shown below in formula I: 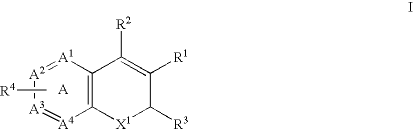
wherein X¹ is selected from O, S, CR^{c}R^{b} and NR^{a};
wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;
wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{b}R^{c} forms a cycloalkyl ring;
wherein R¹ is selected from carboxyl, alkyl, aralkyl, aminocarbonyl, C₁-C₆-alkylsulfonylaminocarbonyl and alkoxycarbonyl;
wherein R² is selected from hydrido, phenyl, thienyl, C₂-C₆-alkynyl, C₁-C₆-alkyl and C₂-C₆-alkenyl;
wherein R³ is selected from C₁-C₃-perfluoroalkyl, chloro, C₁-C₆-alkylthio, C₁-C₆-alkoxy, nitro, phenyl, cyano, cyano-C₁-C₃-alkyl, haloalkyl, alkyl, aralkyl, cycloalkyl, and aryl, wherein haloalkyl, alkyl, aralkyl, cycloalkyl, and aryl each is independently optionally substituted with one or more radicals selected from alkylthio, nitro and alkylsulfonyl;
wherein R⁴ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, O(CF₂)₂ O—, aryloxy, arylthio, arylsulfinyl, heteroaryloxy, aralkyloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₁-alkoxycarbonyl, formyl, C₁-C₆-haloalkylcarbonyl and C₁-C₆-alkylcarbonyl; and
wherein the A ring atoms A¹, A², A³ and A⁴ are independently selected from carbon and nitrogen with the proviso that at least two of A¹, A², A³ and A⁴ are carbon; or
wherein R⁴ together with ring A forms a radical selected from naphthyl, quinolyl, isoquinolyl, quinolizinyl, quinoxalinyl and dibenzofuryl; or an isomer or pharmaceutically acceptable salt thereof.

The meaning of any substituent at any one occurrence in any general chemical formula herein, is independent of its meaning, or any other substituent's meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The number of carbon atoms can also be expressed as “C₁-C₅”, for example. Examples of such radicals include methyl, ethyl, n-propyl, isopropyl, n-butyl, isobutyl, sec-butyl, tert-butyl, pentyl, isoamyl, hexyl, octyl and the, like. The term “alkenyl” refers to an unsaturated, acyclic hydrocarbon radical, linear or branched, in so much as it contains at least one double bond. Unless otherwise noted, such radicals preferably contain from 2 to about 6 carbon atoms, preferably from 2 to about 4 carbon atoms, more preferably from 2 to about 3 carbon atoms. The alkenyl radicals may be optionally substituted with groups as defined below. Examples of suitable alkenyl radicals include propenyl, 2-chloropropylenyl, buten-1yl, isobutenyl, penten-1 yl, 2-methylbuten-1-yl, 3-methylbuten-1-yl, hexen-1-yl, 3-hydroxyhexen-1-yl, hepten-1-yl, octen-1-yl, and the like. The term “alkynyl” refers to an unsaturated, acyclic hydrocarbon radical, linear or branched, in so much as it contains one or more triple bonds, such radicals preferably containing 2 to about 6 carbon atoms, more preferably from 2 to about 3 carbon atoms. The alkynyl radicals may be optionally substituted with groups as described below. Examples of suitable alkynyl radicals include ethynyl, proynyl, hydroxypropynyl, butyn-1-yl, butyn-2-yl, pentyn-1-yl, pentyn-2-yl, 4-methoxypentyn-2-yl, 3-methylbutyn-1-yl, hexyl-1-yl, hexyn-2-yl, hexyn-3-yl, 3,3-dimethylbutyn-1-yl radicals, and the like.

The term “oxo” means a single double-bonded oxygen. The terms “hydrido”, “—H”, or “hydrogen”, denote a single hydrogen atom (H). This hydrido radical may be attached, for example, to an oxygen atom to form a hydroxyl radical, or two hydrido radicals may be attached to a carbon atom to form a methylene (—CH₂—) radical.

The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo radicals may have two or more of the same halo atoms or a combination of different halo radicals and polyhaloalkyl radicals may have more than two of the same halo atoms or a combination of different halo radicals. Likewise, the term “halo”, when it is appended to alkenyl, alkynyl, alkoxy, aryl, cycloalkyl, heteroalkyl, heteroaryl, and the like, includes radicals having mono-, di-, or tri-, halo substitution on one or more of the atoms of the radical.

The term “hydroxyalkyl” embraces linear or branched alkyl radicals having one to about ten carbon atoms any one of which may be substituted with one or more hydroxyl radicals.

The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy. Terms such as “alkoxy(halo)alkyl”, indicate a molecule having a terminal alkoxy that is bound to an alkyl, which is bonded to the parent molecule, while the alkyl also has a substituent halo group in a non-terminal location. In other words, both the alkoxy and the halo group are substituents of the alkyl chain.

The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl. The term “heterocyclyl” means a saturated or unsaturated mono- or multi-ring carbocycle wherein one or more carbon atoms is replaced by N, S, P, or O. This includes, for example, structures such as: 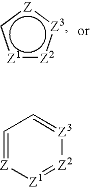
where Z, Z¹, Z², or Z³ is C, S, P, O, or N, with the proviso that one of Z, Z¹, Z², or Z³ is other than carbon, but is not O or S when attached to another Z atom by a double bond or when attached to another O or S atom. Furthermore, the optional substituents are understood to be attached to Z, Z¹, Z², or Z³ only when each is C. The term “heterocycle” also includes fully saturated ring structures, such as piperazinyl, dioxanyl, tetrahydrofuranyl, oxiranyl, aziridinyl, morpholinyl, pyrrolidinyl, piperidinyl, thiazolidinyl, and others. The term “heteroaryl” embraces unsaturated heterocyclic radicals. Examples of unsaturated heterocyclic radicals, also termed “heteroaryl” radicals include thienyl, pyrryl, furyl, pyridyl, pyrimidyl, pyrazinyl, pyrazolyl, oxazolyl, isoxazolyl, imidazolyl, thiazolyl, pyranyl, and tetrazolyl. The term also embraces radicals where heterocyclic radicals are fused with aryl radicals. Examples of such fused bicyclic radicals include benzofuran, benzothiophene, and the like. The terms aryl or heteroaryl, as appropriate, include the following structures: 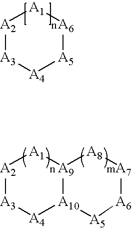
   where:
when n=1, m=1 and A₁-A₈ are each CR^{x} or N, A₉ and A₁₀ are carbon;
when n=0, or 1, and m=0, or 1, one of A₂-A₄ and/or A₅-A₇ is optionally S, O, or NR^{x}, and other ring members are CR^{x} or N, with the proviso that oxygen cannot be adjacent to sulfur in a ring. A₉ and A₁₀ are carbon;
when n is greater than or equal to 0, and m is greater than or equal to 0, 1 or more sets of 2 or more adjacent atoms A₁-A₁₀ are sp3 O, S, NR^{x}, CR^{x}R^{y}, or C═(O or S), with the proviso that oxygen and sulfur cannot be adjacent. The remaining A₁-A₈ are CR^{x} or N, and A₉ and A₁₀ are carbon;
when n is greater than or equal to 0, and m is greater than or equal to 0, atoms separated by 2 atoms (i.e., A₁ and A₄) are sp3 O, S, NR^{x}, CR^{x}R^{y}, and remaining A₁-A₈ are independently CR^{x} or N, and A₉ and A₁₀ are carbon.

The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂—NH₂), which may also be termed an “aminosulfonyl”. The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical.

The terms “carboxy” or “carboxyl”, whether used alone or with other terms, such as “carboxyalkyl”, denotes —CO₂—H. The term “carboxyalkyl” embraces radicals having a carboxyradical as defined above, attached to an alkyl radical. The term “carbonyl”, whether used alone or with other terms, such as “alkylcarbonyl”, denotes —(C═O)—. The term “alkylcarbonyl” embraces radicals having a carbonyl radical substituted with an alkyl radical. An example of an “alkylcarbonyl” radical is CH₃— (CO)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃—C—O—C═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃C—OC(═O)—(CH₂)₂— and —(CH₂)₂ (—O)COCH₃. The terms “amido”, or “carbamyl”, when used alone or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical. The terms “N-alkylamido” and “N,N-dialkylamido” denote amido groups which have been substituted with one alkylradical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-N-hydroxyamidoalkyl” embraces alkylradicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(—NH)—NH₂ radical. The term “cyanoamidin” denotes an —C(—N—CN)—NH₂ radical. The term “heterocycloalkyl” embraces heterocyclic-substituted alkyl radicals such as pyridylmethyl and thienylmethyl.

The terms “aralkyl”, or “arylalkyl” embrace aryl-substituted alkyl radicals such as benzyl, diphenylmethyl, triphenylmethyl, phenethyl, and diphenethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl. The term “alkylthio” embraces radicals containing a linear or branched alkyl radical, of one to ten carbon atoms, attached to a divalent sulfur atom. An example of “alkylthio” is methylthio, (CH₃—S—). The term “alkylsulfinyl” embraces radicals containing a linear or branched alkyl radical, of one to ten carbon atoms, attached to a divalent —S(—O)— atom. The terms “N-alkylamino” and “N,N-dialkylamino” denote amino groups which have been substituted with one alkyl radical and with two alkyl radicals, respectively.

The term “acyl”, whether used alone, or within a term such as “acylamino”, denotes a radical provided by the residue after removal of hydroxyl from an organic acid. The term “acylamino” embraces an amino radical substituted with an acyl group. An examples of an “acylamino” radical is acetylamino (CH₃—C(═O)—NH—).

In the naming of substituent groups for general chemical structures, the naming of the chemical components of the group is typically from the terminal group-toward the parent compound unless otherwise noted, as discussed below. In other words, the outermost chemical structure is named first, followed by the next structure in line, followed by the next, etc. until the structure that is connected to the parent structure is named. For example, a substituent group having a structure such as: 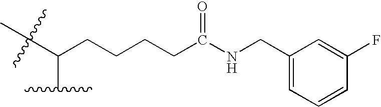
may be referred to generally as a “haloarylalkylaminocarboxylalkyl”. An example of one such group would be fluorophenylmethylcarbamylpentyl. The bonds having wavy lines through them represent the parent structure to which the alkyl is attached.

Substituent groups may also be named by reference to one or more “R” groups. The structure shown above would be included in a description, such as, “—C₁-C₆-alkyl-COR^{u}, where R^{u} is defined to include —NH—C₁-C₄-alkylaryl-R^{y}, and where R^{y} is defined to include halo. In this scheme, atoms having an “R” group are shown with the “R” group being the terminal group (i.e., furthest from the parent). In a term such as “C(R^{x})₂”, it should be understood that the two R^{x} groups can be the same, or they can be different if R^{x} is defined as having more than one possible identity.

Examples of chromene Cox-2 inhibitors that are suitable for use with the methods and compositions of the present invention include any one or more of:

6-nitro-2-trifluoromethyl-2H-1-benzopyran-3-carboxylic acid; 6-chloro-8-methyl-2-trifluoromethyl-2H-1-benzopyran-3-carboxylic acid; (S)-6-chloro-7-(1,1-dimethylethyl)-2-trifluoromethyl-2H-1-benzopyran-3-carboxylic acid; 2-trifluoromethyl-2H-naphthol[2,3-b]pyran-3-carboxylic acid; 6-chloro-7-(4-nitrophenoxy)-2-(trifluoromethyl)-2H-1-benzopyran-3-carboxylic acid; (S)-6,8-dichloro-2-(trifluoromethyl)-2H-1-benzopyran-3-carboxylic acid; 6-chloro-2-(trifluoromethyl)-4-phenyl-2H-1-benzopyran-3-carboxylic acid; 6-(4-hydroxybenzoyl)-2-(trifluoromethyl)-2H-1-benzopyran-3-carboxylic acid; 2-(trifluoromethyl)-6-[(trifluoromethyl)thiol]-2H-1-benzothiopyran-3-carboxylic acid; 6,8-dichloro-2-trifluoromethyl-2H-1-benzothiopyran-3 carboxylic acid; 6-(1,1-dimethylethyl)-2-(trifluoromethyl)-2H-1-benzothiopyran-3-carboxylic acid; 6,7-difluoro-1,2-dihydro-2-(trifluoromethyl)-3-quinolinecarboxylic acid; 6-chloro-1,2-dihydro-1-methyl-2-(trifluoromethyl)-3-quinolinecarboxylic acid; 6-chloro-2-(trifluoromethyl)-1,2-dihydro[1,8]naphthyridine-3-carboxylic acid; (S)-6-chloro-1,2-dihydro-2-(trifluoromethyl)-3-quinolinecarboxylic acid; (2S)-6,8-dimethyl-2-(trifluoromethyl)-2H-ch romene-3-carboxylic acid; (2S)-8-ethyl-6-(trifluoromethoxy)-2-(trifluoromethyl)-2H-chromene-3-carboxylic acid; (2S)-6-chloro-5,7-dimethyl-2-(trifluoromethyl)-2H-chromene-3-carboxylic acid; 6-chloro-2-trifluoromethyl-2H-1-benzopyran-3-carboxylic acid; 6-chloro-7-methyl-2-trifluoromethyl-2H-1-benzopyran-3-carboxylic acid; 8-(1-methylethyl)-2-trifluoromethyl-2H-1-benzopyran-3-carboxylic acid; 6-chloro-7-(1,1-dimethylethyl)-2-trifluoromethyl-2H-1-benzopyran-3-carboxylic acid; 6-chloro-8-(1-methylethyl)-2-trifluoromethyl-2H-1-benzopyran-3-carboxylic acid; 2-trifluoromethyl-3H-naphthopyran-3-carboxylic acid; 7-(1,1-dimethylethyl)-2-trifluoromethyl-2H-1-benzopyran-3-carboxylic acid; 6-bromo-2-trifluoromethyl-2H-1-benzopyran-3-carboxylic acid; 8-chloro-2-trifluoromethyl-2H-1-benzopyran-3-carboxylic acid; 6-trifluoromethoxy-2-trifluoromethyl-2H-1-benzopyran-3-carboxylic acid; 5,7-dichloro-2-trifluoromethyl-2H-1-benzopyran-3-carboxylic acid; 8-phenyl-2-trifluoromethyl-2H-1-benzopyran-3-carboxylic acid; 7,8-dimethyl-2-trifluoromethyl-2H-1-benzopyran-3-carboxylic acid; 6,8-bis(dimethylethyl)-2-trifluoromethyl-2H-1-benzopyran-3-carboxylic acid; 7-(1-methylethyl)-2-trifluoromethyl-2H-1-benzopyran-3-carboxylic acid; 7-phenyl-2-trifluoromethyl-2H-1-benzopyran-3-carboxylic acid; 6-chloro-7-ethyl-2-trifluoromethyl-2H-1-benzopyran-3-carboxylic acid; 6-chloro-8-ethyl-2-trifluoromethyl-2H-1-benzopyran-3-carboxylic acid; 6-chloro-7-phenyl-2-trifluoromethyl-2H-1-benzopyran-3-carboxylic acid; 6,7-dichloro-2-trifluoromethyl-2H-1-benzopyran-3-carboxylic acid; 6,8-dichloro-2-trifluoromethyl-2H-1-benzopyran-3-carboxylic acid; 2-trifluoromethyl-3H-naptho[2,1-b]pyran-3-carboxylic acid; 6-chloro-8-methyl-2-trifluoromethyl-2H-1-benzopyran-3-carboxylic acid; 8-chloro-6-methyl-2-trifluoromethyl-2H-1-benzopyran-3-carboxylic acid; 8-chloro-6-methoxy-2-trifluoromethyl-2H-1-benzopyran-3-carboxylic acid; 6-bromo-8-chloro-2-trifluoromethyl-2H-1-benzopyran-3-carboxylic acid; 8-bromo-6-fluoro-2-trifluoromethyl-2H-1-benzopyran-3-carboxylic acid; 8-bromo-6-methyl-2-trifluoromethyl-2H-1-benzopyran-3-carboxylic acid; 8-bromo-5-fluoro-2-trifluoromethyl-2H-1-benzopyran-3-carboxylic acid; 6-chloro-8-fluoro-2-trifluoromethyl-2H-1-benzopyran-3-carboxylic acid; 6-bromo-8-methoxy-2-trifluoromethyl-2H-1-benzopyran-3-carboxylic acid; 6-[[(phenylmethyl)amino]sulfonyl]-2-trifluoromethyl-2H-1-benzopyran-3-carboxylic acid; 6-[(dimethylamino)sulfonyl]-2-trifluoromethyl-2H-1-benzopyran-3-carboxylic acid; 6-[(methylamino)sulfonyl]-2-trifluoromethyl-2H-1-benzopyran-3-carboxylic acid; 6-[(4-morpholino)sulfonyl]-2-trifluoromethyl-2H-1-benzopyran-3-carboxylic acid; 6-[(1,1-dimethylethyl)aminosulfonyl]-2-trifluoromethyl-2H-1-benzopyran-3-carboxylic acid; 6-[(2-methylpropyl)aminosulfonyl]-2-trifluoromethyl-2H-1-benzopyran-3-carboxylic acid; 6-methylsulfonyl-2-trifluoromethyl-2H-1-benzopyran-3-carboxylic acid; 8-chloro-6-[[(phenylmethyl)amino]sulfonyl]-2-trifluoromethyl-2H-1-benzopyran-3-carboxylic acid; 6-phenylacetyl-2-trifluoromethyl-2H-1-benzopyran-3-carboxylic acid; 6,8-dibromo-2-trifluoromethyl-2H-1-benzopyran-3-carboxylic acid; 8-chloro-5,6-dimethyl-2-trifluoromethyl-2H-1-benzopyran-3-carboxylic acid; 6,8-dichloro-(S)-2-trifluoromethyl-2H-1-benzopyran-3-carboxylic acid; 6-benzylsulfonyl-2-trifluoromethyl-2H-1-benzopyran-3-carboxylic acid; 6-[[N-(2-furylmethyl)amino]sulfonyl]-2-trifluoromethyl-2H-1-benzopyran-3-carboxylic acid; 6-[[N-(2-phenylethyl)amino]sulfonyl]-2-trifluoromethyl-2H-1-benzopyran-3-carboxylic acid; 6-iodo-2-trifluoromethyl-2H-1-benzopyran-3-carboxylic acid; 7-(1,1-dimethylethyl)-2-pentafluoroethyl-2H-1-benzopyran-3-carboxylic acid; 6-chloro-2-trifluoromethyl-2H-1-benzothiopyran-3-carboxylic acid; 6-chloro-2-trifluoromethyl-2H-1-benzopyran-3-carboxylic acid; (S)-6-chloro-2-trifluoromethyl-2H-1-benzopyran-3-carboxylic acid; 6-chloro-7-(1,1-dimethylethyl)-2-trifluoromethyl-2H-1-benzopyran-3-carboxylic acid; (S)-6-chloro-7-(1,1-dimethylethyl)-2-(trifluoromethyl)-2H-1-benzopyran-3-carboxylic acid; 6-trifluoromethoxy-2-trifluoromethyl-2H-1-benzopyran-3-carboxylic acid; (S)-6-trifluoromethoxy-2-trifluoromethyl-2H-1-benzopyran-3-carboxylic acid; 6-formyl-2-(trifluoromethyl)-2H-1-benzopyran-3-carboxylic acid; 6-(difluoromethyl)-2-(trifluoromethyl)-2H-1-benzopyran-3-carboxylic acid; 6,8-dichloro-7-methyl-2-(trifluoromethyl)-2H-1-benzopyran-3-carboxylic acid; 6,8-dichloro-2-trifluoromethyl-2H-1-benzopyran-3-carboxylic acid; (S)-6,8-dichloro-2-(trifluoromethyl)-2H-1-benzopyran-3-carboxylic acid; 6-chloro-1,2-dihydro-2-(trifluoromethyl)-3-quinolinecarboxylic acid; (S)-6-chloro-1,2-dihydro-2-(trifluoromethyl)-3-quinolinecarboxylic acid; 6,8-dichloro-1,2-dihydro-2-(trifluoromethyl)-3-quinolinecarboxylic acid; 7-(1,1-dimethylethyl)-2-trifluoromethyl-2H-1-benzopyran-3-carboxylic acid; 6,7-dichloro-2-trifluoromethyl-2H-1-benzopyran-3-carboxylic acid; 5,6-dichloro-2-(trifluoromethyl)-2H-1-benzopyran-3-carboxylic acid; 2,6-bis(trifluoromethyl)-2H-1-benzopyran-3-carboxylic acid; 5,6,7-trichloro-2-(trifluoromethyl)-2H-1-benzopyran-3-carboxylic acid; 6,7,8-trichloro-2-(trifluoromethyl)-2H-1-benzopyran-3-carboxylic acid; 6-iodo-1,2-dihydro-2-(trifluoromethyl)-3-quinolinecarboxylic acid; 6-bromo-1,2-dihydro-2-(trifluoromethyl)-3-quinolinecarboxylic acid; 6-chloro-7-methyl-2-(trifluoromethyl)-2H-1-benzothiopyran-3-carboxylic acid; 6,8-dichloro-2-trifluoromethyl-2H-1-benzothiopyran-3-carboxylic acid; prod rugs thereof, salts thereof; isomers thereof; and/or mixtures thereof.

Further examples of preferred chromene Cox-2 inhibitors include (S)-6-chloro-7-(1,1-dimethylethyl)-2-(trifluoromethyl)-2H-1-benzopyran-3-carboxylic acid, (2S)-6,8-dimethyl-2-(trifluoromethyl)-2H-chromene-3-carboxylic acid, (2S)-6-chloro-8-methyl-2-(trifluoromethyl)-2H-ch romene-3-carboxylic acid, (2S)-8-ethyl-6-(trifluoromethoxy)-2-(trifluoromethyl)-2H-chromene-3-carboxylic acid, (S)-6,8-dichloro-2-(trifluoromethyl)-2H-1-benzopyran-3-carboxylic acid, (2S)-6-chloro-5,7-dimethyl-2-(trifluoromethyl)-2H-ch romene-3-carboxylic acid, prodrugs thereof, salts thereof, isomers thereof, and/or mixtures thereof.

In another embodiment of the invention, the Cox-2 inhibitor can be selected from the class of tricyclic Cox-2 selective inhibitors represented by the general structure of formula II: 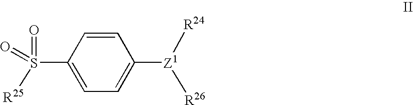
wherein:
Z¹ is selected from the group consisting of partially unsaturated or unsaturated heterocyclyl and partially unsaturated or unsaturated carbocyclic rings;
R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R²⁵ is selected from the group consisting of methyl or amino; and
R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; prodrugs thereof; salts thereof; isomers thereof; and/or mixtures thereof.

In one embodiment of the invention, the tricyclic Cox-2 selective inhibitor comprises at least one compound chosen from celecoxib, parecoxib, deracoxib, valdecoxib, lumiracoxib, etoricoxib, rofecoxib, tilmacoxib, cimicoxib, prodrugs thereof, salts thereof, isomers thereof, and/or mixtures thereof.

In another embodiment of the invention, the Cox-2 selective inhibitor represented by the above Formula II is chosen from those compounds, illustrated in Table 1, which includes celecoxib (B-1), valdecoxib (B-2), deracoxib (B-3), rofecoxib (B-4), etoricoxib (MK-663; B-5), tilmacoxib (JTE-522) (B-6), cimicoxib (B-7), prodrugs thereof, salts thereof, isomers thereof, and/or mixtures thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-9 (U.S. Pat. No. 5,840,924); compound B-10 (WO 00/25779); cimicoxib (4-[4-chloro-5-(3-fluoro-4-methoxyphenyl)-1H-imidzol-1-yl]benzenesulfonamide—CAS RN 265114-23-6); tilmacoxib (4-(4-cyclohexyl-2-methyloxazol-5-yl)-2-fluorobenzenesulfonamide—JTE-522, CAS 180200-68-4); and etoricoxib (CAS RN 202409-33-4, MK-663, SC-86218, and in WO 98/03484).

TABLE 1
Examples of Tricyclic Cox-2 Selective Inhibitors

| Compound | Common | Chemical |
|---|---|---|
| No. | name | name |
| B-1 | celecoxib | 4-[5-(4-methylphenyl)-3-(trifluoromethyl)-1H- |
| | | pyrazol-1-yl] benzenesulfonamide |
| B-2 | valdecoxib | 4-(5-methyl-3-phenyl-4-isoxazolyl) |
| | | benzenesulfonamide |
| B-3 | deracoxib | 4-[3-(difluoromethyl)-5-(3-fluoro-4- |
| | | methoxyphenyl)-1H-pyrazol-1- |
| | | yl]benzenesulfonamide |
| B-4 | rofecoxib | 4-[4-(methylsulfonyl)phenyl]-3-phenyl-2(5H)- |
| | | furanone |
| B-5 | etoricoxib | 2,3′-bipyridine, 5-chloro-6′-methyl-3-[4- |
| | | [methylsulfonyl]phenyl]-; or [2] 5-chloro-6′- |
| | | methyl-3-[p-[methylsulfonyl]phenyl]-2,3′- |
| | | bipyridine |
| B-6 | tilmacoxib | 4-(4-cyclohexyl-2-methyloxazol-5-yl)-2- |
| | | fluorobenzenesulfonamide |
| B-7 | cimicoxib | 4-[4-chloro-5-(3-fluoro-4-methoxyphenyl)-1H- |
| | | imidzol-1-yl]benzenesulfonamide |
| B-8 | parecoxib | N-[[p-(5-methyl-3-phenyl-4-isox- |
| | | azolyl)phenyl]sulfonyl]propionamide |

In yet another embodiment of the invention, the Cox-2 selective inhibitor is selected from the group consisting of celecoxib, valdecoxib, rofecoxib, etoricoxib, tilmacoxib, cimicoxib, etoricoxib, deracoxib, parecoxib, prodrugs thereof, salts thereof, isomers thereof, and/or mixtures thereof. Even more preferred still is that the Cox-2 selective inhibitor is celecoxib.

In another embodiment, the tricyclic Cox-2 selective inhibitor, parecoxib (B-8), N-[[4-(5-methyl-3-phenyl-4-isoxazo-lyl)phenyl]sulfonyl]-; or (2) N-[[p-(5-methyl-3-phenyl-4-isox-azolyl)phenyl]sulfonyl]propionamide; CAS No. 198470-84-7 (See, U.S. Pat. No. 5,932,598), which is a therapeutically effective prodrug of the tricyclic Cox-2 selective inhibitor valdecoxib, compound B-2, (See, U.S. Pat. No. 5,633,272), may be advantageously employed as the Cox-2 inhibitor of the present invention.

A preferred form of parecoxib is sodium parecoxib, which is available as Dynastat®.

Another tricyclic Cox-2 selective inhibitor useful in the present invention is the compound ABT-963, having the structure: 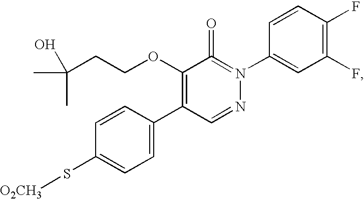
which has been previously described in International Publication Number WO 00/24719.

In a further embodiment of the invention, the Cox-2 inhibitor can be selected from the class of phenylacetic acid derivative Cox-2 selective inhibitors represented by the general structure of formula III: 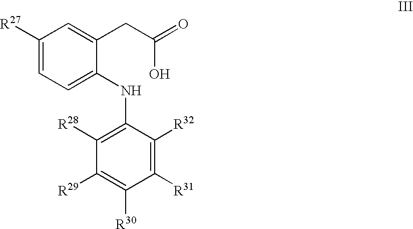
wherein:
R²⁷ is methyl, ethyl, or propyl;
R²⁸ is chloro or fluoro;
R²⁹ is hydrogen, fluoro, or methyl;
R³⁰ is hydrogen, fluoro, chloro, methyl, ethyl, methoxy, ethoxy or hydroxyl;
R³¹ is hydrogen, fluoro, or methyl; and
R³² is chloro, fluoro, trifluoromethyl, methyl, or ethyl, provided that R²⁸, R²⁹, R³⁰ and R³¹ are not all fluoro when R²⁷ is ethyl and R³⁰ is H.

An exemplary phenylacetic acid derivative Cox-2 selective inhibitor that is described in WO 99/11605 is a compound that has the structure shown in formula III,

wherein:

R²⁷ is ethyl;
R²⁸ and R³⁰ are chloro;
R²⁹ and R³¹ are hydrogen; and
R³² is methyl.

Another phenylacetic acid derivative Cox-2 selective inhibitor is a compound that has the structure shown in formula III,

wherein:

R²⁷ is propyl;
R²⁸ and R³⁰ are chloro;
R²⁹ and R³¹ are methyl; and
R³² is ethyl.

Another phenylacetic acid derivative Cox-2 selective inhibitor that is disclosed in WO 02/20090 is a compound that is referred to as COX-189 (also termed lumiracoxib; CAS Reg. No. 220991-20-8), having the structure shown in formula III,

wherein:

R²⁷ is methyl;
R²⁸ is fluoro;
R³² is chloro; and
R²⁹, R³⁰, and R³¹ are hydrogen.

Compounds having a structure similar to that shown in formula VIII, that can serve as the Cox-2 selective inhibitor of the present invention, are described in U.S. Pat. Nos. 6,451,858, 6,310,099, 6,291,523, and 5,958,978.

In certain aspects of the present invention, the Cox-2 selective inhibitor may be a Cox-2 selective inhibitor that is other than any tricyclic Cox-2 selective inhibitor described by formula II. For example, the Cox-2 selective inhibitor may be a chromene Cox-2 inhibitor, which is a class of Cox-2 selective inhibitor that is other than a tricylic Cox-2 selective inhibitor. Likewise, the Cox-2 selective inhibitor may be any compound described by formula III, such as lumiracoxib, which is other than a tricyclic Cox-2 selective inhibitor. Thus, in some embodiments, the present invention encompasses any Cox-2 selective inhibitor that is other than a tricyclic Cox-2 selective inhibitor that is described by formula II, whether such a Cox-2 selective inhibitor is now known or later developed.

In other aspects of the present invention, the Cox-2 selective inhibitor may be at least one compound or class of compounds chosen from Table 2, isomers thereof, salts thereof, and/or mixtures thereof. However, the present invention should not be construed as being limited to any particular one of the Cox-2 selective inhibitors described herein. Indeed, it should be understood that the present invention encompasses any compound that can be shown to act as an inhibitor of the Cox-2 enzyme, whether such a compound is now known, later developed, or even later recognized as having Cox-2 inhibitory activity.

TABLE 2
Additional Cox-2 Selective Inhibitors

| | Generic Name/ | Trade | | | | |
|---|---|---|---|---|---|---|
| No. | Compound Name | Name(s) | Drug Class/Mode of Action | Dose | Manufacturer | Reference |
| B11 | Nimesulide | | | | | |
| B12 | Flosulide | | | | | |
| B13 | NS-398 | | | | | CAS RN 123653-11-2 |
| | N-(2- | | | | | Yoshimi, N. et al., in |
| | cyclohexyloxynitrophenyl) | | | | | Japanese J. Cancer |
| | methane sulfonamide | | | | | Res., 90(4): 406-412 |
| | | | | | | (1999) |
| B14 | L-745337 | | | | | |
| B15 | RWJ-63556 | | | | | Kirchner et al., in J |
| | | | | | | Pharmacol Exp Ther |
| | | | | | | 282, 1094-1101 |
| | | | | | | (1997) |
| B16 | L-784512 | | | | | |
| B17 | N-(2- | | diarylmethylidenefuran | | | U.S. Pat. No. |
| | cyclohexyloxynitrophenyl)methane | | derivatives | | | 6,180,651 |
| | sulfonamide, and | | | | | |
| | (E)-4-[(4- | | | | | |
| | methylphenyl)(tetrahydro-2- | | | | | |
| | oxo-3-furanylidene) | | | | | |
| | methyl]benzenesulfonamide | | | | | |
| B18 | Darbufelone | | | | Pfizer | |
| B19 | CS-502 | | | | Sankyo | |
| B20 | LAS 34475 | | | | Almirall | |
| | | | | | Profesfarma | |
| B21 | LAS 34555 | | | | Almirall | |
| | | | | | Profesfarma | |
| B22 | S-33516 | | | | Servier | |
| B23 | SD 8381 | | | | Pharmacia | U.S. Pat. No. |
| | | | | | | 6,034,256 |
| B24 | BMS-347070 | | | | Bristol Myers | U.S. Pat. No. |
| | | | | | Squibb | 6,180,651 |
| B25 | MK-966 | | | | Merck | |
| B26 | L-783003 | | | | Merck | |
| B27 | T-614 | | | | Toyama | |
| B28 | D-1367 | | | | Chiroscience | |
| B29 | L-748731 | | | | Merck | |
| B30 | CT3 | | | | Atlantic | |
| | | | | | Pharmaceutical | |
| B31 | CGP-28238 | | | | Novartis | |
| B32 | BF-389 | | | | Biofor/Scherer | |
| B33 | GR-253035 | | | | Glaxo Wellcome | |
| B34 | 6-dioxo-9H-purin-8-yl- | | | | Glaxo Wellcome | |
| | cinnamic acid | | | | | |
| B35 | S-2474 | | | | Shionogi | |
| B36 | | | Multibinding compounds | | | U.S. Pat. No. |
| | | | containing from 2 to 10 ligands | | | 6,395,724 |
| | | | covanlently attached to one or | | | |
| | | | more linkers | | | |
| B37 | | | Conjugated linoleic acid | | | U.S. Pat. No. |
| | | | derivatives | | | 6,077,868 |
| B38 | | | Heterocyclic aromatic oxazole | | | U.S. Pat. Nos. |
| | | | compounds | | | 5,994,381 and |
| | | | | | | 6,362,209 |
| B39 | | | Miscellaneous compounds | | | U.S. Pat. Nos. |
| | | | | | | 6,596,736, 6,369,275, |
| | | | | | | 6,127,545, 6,130,334, |
| | | | | | | 6,204,387, 6,071,936, |
| | | | | | | 6,001,843 and |
| | | | | | | 6,040,450 |
| B40 | | | Diarylbenzopyran derivatives | | | U.S. Pat. No. |
| | | | | | | 6,340,694 |
| B41 | | | 1-(4-sulfamylaryl)-3-substituted- | | | U.S. Pat. No. |
| | | | 5-aryl-2-pyrazolines | | | 6,376,519 |
| B42 | | | Heterocycle compounds | | | U.S. Pat. No. |
| | | | | | | 6,153,787 |
| B43 | | | 2,3,5-trisubstituted pyridines | | | U.S. Pat. No. |
| | | | | | | 6,046,217 |
| B44 | | | Diaryl bicyclic heterocycles | | | U.S. Pat. No. |
| | | | | | | 6,329,421 |
| B45 | | | Salts of 5-amino or substituted | | | U.S. Pat. No. |
| | | | amino 1,2,3-triazole | | | 6,239,137 |
| | | | compounds | | | |
| B46 | | | Pyrazole derivatives | | | U.S. Pat. No. |
| | | | | | | 6,136,831 |
| B47 | | | Substituted derivatives of | | | U.S. Pat. No. |
| | | | benzosulphonamides | | | 6,297,282 |
| B48 | 3-phenyl-4-(4- | | Phenyl heterocycles | | | U.S. Pat. Nos. |
| | (methylsulfonyl)phenyl)-2- | | | | | 5,474,995 and |
| | (2H)-furanone | | | | | 6,239,173 |
| B49 | | | Bicycliccarbonyl indole | | | U.S. Pat. No. |
| | | | compounds | | | 6,303,628 |
| B50 | | | Benzimidazole compounds | | | U.S. Pat. No. |
| | | | | | | 6,310,079 |
| B51 | | | Indole compounds | | | U.S. Pat. No. |
| | | | | | | 6,300,363 |
| B52 | | | Aryl phenylhydrazides | | | U.S. Pat. No. |
| | | | | | | 6,077,869 |
| B53 | | | 2-aryloxy, 4-aryl furan-2-ones | | | U.S. Pat. No. |
| | | | | | | 6,140,515 |
| B54 | | | Bisaryl compounds | | | U.S. Pat. No. |
| | | | | | | 5,994,379 |
| B55 | | | 1,5-diarylpyrazoles | | | U.S. Pat. No. |
| | | | | | | 6,028,202 |
| B56 | | | 2-substituted imidazoles | | | U.S. Pat. No. |
| | | | | | | 6,040,320 |
| B57 | | | 1,3- and 2,3-diarylcycloalkano | | | U.S. Pat. No. |
| | | | and cycloalkeno pyrazoles | | | 6,083,969 |
| B58 | | | Esters derived from | | | U.S. Pat. No. |
| | | | indolealkanols and novel | | | 6,306,890 |
| | | | amides derived from | | | |
| | | | indolealkylamides | | | |
| B59 | | | Pyridazinone compounds | | | U.S. Pat. No. |
| | | | | | | 6,307,047 |
| B60 | | | Benzosulphonamide derivatives | | | U.S. Pat. No. |
| | | | | | | 6,004,948 |
| B61 | | | Methanesulfonyl-biphenyl | | | U.S. Pat. No. |
| | | | derivatives | | | 6,583,321 |
| B62 | | | 1H-indole derivatives | | | U.S. Pat. No. |
| | | | | | | 6,599,929 |
| B63 | N-(2-hydroxyethyl)-4-[5-(4- | | Certain prodrugs of Cox-2 | | | U.S. Pat. Nos. |
| | methylphenyl)-3- | | inhibitors | | | 6,436,967 and |
| | (trifluoromethyl)-1H-pyrazol- | | | | | 6,613,790 |
| | 1-yl]benzenesulfonamide, | | | | | |
| | N,N-bis(2-hydroxyethyl)-4- | | | | | |
| | [5-(4-methylphenyl)-3- | | | | | |
| | (trifluoromethyl)-1H-pyrazol- | | | | | |
| | 1-yl]benzenesulfonamide | | | | | |
| B64 | | | Sulfamoylheteroaryl pyrazole | | | U.S. Pat. No. |
| | | | compounds | | | 6,583,321 |
| B65 | | | Heteroaryl substituted amidinyl | | | U.S. Pat. No. |
| | | | and imidazolyl compounds | | | 6,555,563 |
| B66 | | | Substituted hydroxamic acid | | | U.S. Pat. Nos. |
| | | | derivatives | | | 6,432,999, 6,512,121, |
| | | | | | | and 6,515,014 |
| B67 | | | Pyrazolopyridine compounds | | | U.S. Pat. No. |
| | | | | | | 6,498,166 |
| B68 | | | 4,5-diaryl-3(2H)-furanone | | | U.S. Pat. No. |
| | | | derivatives | | | 6,492,416 |
| B69 | | | 2-phenyl-1,2-benzisoselenazol- | | | U.S. Pat. No. |
| | | | 3(2H)-one derivatives and 2- | | | 6,492,416 |
| | | | phenylcarbomyl-phenylselenyl | | | |
| | | | derivatives | | | |
| B70 | | | Pyrones | | | U.S. Pat. No. |
| | | | | | | 6,465,509 |
| B71 | | | Organically synthesized or | | | U.S. Published |
| | | | purified from plant sources, | | | Application No. |
| | | | free-B-ring flavanoids | | | 2003/0165588 |
| B72 | | | Heterocyclo-alkylsulfonyl | | | European Patent |
| | | | pyrazoles | | | Application No. EP |
| | | | | | | 1312367 |
| B73 | | | 2-phenylpyran-4-one | | | U.S. Pat. No. |
| | | | derivatives | | | 6,518,303 |
| B74 | | | Sulfonylphenylpyrazoles | | | U.S. Pat. No. |
| | | | | | | 6,472,416 |
| B75 | | | 2,3-diaryl-pyrazolo[1,5- | | | U.S. Pat. No. |
| | | | b]pyridazines | | | 6,451,794 |
| B76 | | | (Methylsulfonyl)phenyl | | | U.S. Pat. Nos. |
| | | | furanones | | | 6,169,188, 6,020,343, |
| | | | | | | and 5,981,576 |
| B77 | | | Diaryl-2-(5H)-furanones | | | U.S. Pat. No. |
| | | | | | | 6,222,048 |
| B78 | | | 3,4-diaryl-2-hydroxy-2,5- | | | U.S. Pat. No. |
| | | | dihydrofurans | | | 6,057,319 |
| B79 | | | Carbocyclic sulfonamides | | | U.S. Pat. No. |
| | | | | | | 6,046,236 |
| B80 | | | Oxazole derivatives | | | U.S. Pat. Nos. |
| | | | | | | 6,002,014 and |
| | | | | | | 5,945,539 |
| B81 | | | C-nitroso compounds | | | U.S. Pat. Nos. |
| | | | | | | 6,359,182 and |
| | | | | | | 6,538,116 |
| B82 | | | Substituted pyridines | | | U.S. Published |
| | | | | | | Application No. |
| | | | | | | 2003/0065011 |
| B83 | | | Substituted indole derivatives | | | U.S. Published |
| | | | | | | Application No. |
| | | | | | | 2003/0207897 |
| B84 | meloxicam | | | | | CAS registry number |
| | | | | | | 71125-38-7 |
| B85 | RS 57067 | | | | | CAS registry number |
| | 6-[[5-(4-chlorobenzoyl)-1,4- | | | | | 179382-91-3 |
| | dimethyl-1H-pyrrol-2- | | | | | |
| | yl]methyl]-3(2H)- | | | | | |
| | pyridazinone | | | | | |
| B86 | PMI-001 | | Tricyclic Cox-2 inhibitor - | | Phytomedics | |
| | | | botanical extract | | | |
| B87 | 644784 | | Tricyclic Cox-2 inhibitor | | GlaxoSmithKline | |
| B88 | CS-706 (R-109339) | | Tricyclic Cox-2 inhibitor | | Sankyo | |
| B89 | PAC-10549, PAC-10649 | | Non-tricyclic Cox-2 inhibitor | | Pacific, Corp. | |

Examples of specific compounds that are useful as Cox-2 selective inhibitors include, without limitation:

8-acetyl-3-(4-fluorophenyl)-2-(4-methylsulfonyl)phenyl-imidazo(1,2-a)pyridine; 5,5-dimethyl-4-(4-methylsulfonyl)phenyl-3-phenyl-2-(5H)-furanone; 5-(4-fluorophenyl)-1-[4-(methylsulfonyl)phenyl]-3-(trifluoromethyl)pyrazole; 4-(4-fluorophenyl)-5-[4-(methylsulfonyl)phenyl]-1-phenyl-3-(trifluoromethyl)pyrazole; 4-(5-(4-chlorophenyl)-3-(4-methoxyphenyl)-1H-pyrazol-1-yl)benzenesulfonamide; 4-(3,5-bis(4-methylphenyl)-1H-pyrazol-1-yl)benzenesulfonamide; 4-(5-(4-chlorophenyl)-3-phenyl-1H-pyrazol-1-yl)benzenesulfonamide; 4-(3,5-bis(4-methoxyphenyl)-1H-pyrazol-1-yl)benzenesulfonamide; 4-(5-(4-chlorophenyl)-3-(4-methylphenyl)-1H-pyrazol-1-yl)benzenesulfonamide; 4-(5-(4-chlorophenyl)-3-(4-nitrophenyl)-1H-pyrazol-1-yl)benzenesulfonamide; 4-(5-(4-chlorophenyl)-3-(5-chloro-2-thienyl)-1H-pyrazol-1-yl)benzenesulfonamide; 4-(4-chloro-3,5-diphenyl-1H-pyrazol-1-yl)benzenesulfonamide; 4-[5-(4-chlorophenyl)-3-(trifluoromethyl)-1H-pyrazol-1-yl]benzenesulfonamide; 4-[5-phenyl-3-(trifluoromethyl)-1H-pyrazol-1-yl]benzenesulfonamide; 4-[5-(4-fluorophenyl)-3-(trifluoromethyl)-1H-pyrazol-1-yl]benzenesulfonamide; 4-[5-(4-methoxyphenyl)-3-(trifluoromethyl)-1H-pyrazol-1-yl]benzenesulfonamide; 4-[5-(4-chlorophenyl)-3-(difluoromethyl)-1H-pyrazol-1-yl]benzenesulfonamide; 4-[5-(4-methylphenyl)-3-(trifluoromethyl)-1H-pyrazol-1-yl]benzenesulfonamide; 4-[4-chloro-5-(4-chlorophenyl)-3-(trifluoromethyl)-1H-pyrazol-1-yl]benzenesulfonamide; 4-[3-(difluoromethyl)-5-(4-methylphenyl)-1H-pyrazol-1-yl]benzenesulfonamide; 4-[3-(difluoromethyl)-5-phenyl-1H-pyrazol-1-yl]benzenesulfonamide; 4-[3-(difluoromethyl)-5-(4-methoxyphenyl)-1H-pyrazol-1-yl]benzenesulfonamide; 4-[3-cyano-5-(4-fluorophenyl)-1H-pyrazol-1-yl]benzenesulfonamide; 4-[3-(difluoromethyl)-5-(3-fluoro-4-methoxyphenyl)-1H-pyrazol-1-yl]benzenesulfonamide; 4-[5-(3-fluoro-4-methoxyphenyl)-3-(trifluoromethyl)-1H-pyrazol-1-yl]benzenesulfonamide; 4-[4-chloro-5-phenyl-1H-pyrazol-1-yl]benzenesulfonamide; 4-[5-(4-chlorophenyl)-3-(hydroxymethyl)-1H-pyrazol-1-yl]benzenesulfonamide; 4-[5-(4-(N,N-dimethylamino)phenyl)-3-(trifluoromethyl)-1H-pyrazol-1-yl]benzenesulfonamide; 5-(4-fluorophenyl)-6-[4-(methylsulfonyl)phenyl]spiro[2.4]hept-5-ene; 4-[6-(4-fluorophenyl)spiro[2.4]hept-5-en-5-yl]benzenesulfonamide; 6-(4-fluorophenyl)-7-[4-(methylsulfonyl)phenyl]spiro[3.4]oct-6-ene; 5-(3-chloro-4-methoxyphenyl)-6-[4-(methylsulfonyl)phenyl]spiro[2.4]hept-5-ene; 4-[6-(3-chloro-4-methoxyphenyl)spiro[2.4]hept-5-en-5-yl]benzenesulfonamide; 5-(3,5-dichloro-4-methoxyphenyl)-6-[4-(methylsulfonyl)phenyl]spiro[2.4]hept-5-ene; 5-(3-chloro-4-fluorophenyl)-6-[4-(methylsulfonyl)phenyl]spiro[2.4]hept-5-ene; 4-[6-(3,4-dichlorophenyl)spiro[2.4]hept-5-en-5-yl]benzenesulfonamide; 2-(3-chloro-4-fluorophenyl)-4-(4-fluorophenyl)-5-(4-methylsulfonylphenyl)thiazole; 2-(2-chlorophenyl)-4-(4-fluorophenyl)-5-(4-methylsulfonylphenyl)thiazole; 5-(4-fluorophenyl)-4-(4-methylsulfonylphenyl)-2-methylthiazole; 4-(4-fluorophenyl)-5-(4-methylsulfonylphenyl)-2-trifluoromethylthiazole; 4-(4-fluorophenyl)-5-(4-methylsulfonylphenyl)-2-(2-thienyl)thiazole; 4-(4-fluorophenyl)-5-(4-methylsulfonylphenyl)-2-benzylaminothiazole; 4-(4-fluorophenyl)-5-(4-methylsulfonylphenyl)-2-(1-propylamino)thiazole; 2-[(3,5-dichlorophenoxy)methyl)-4-(4-fluorophenyl)-5-[4-(methylsulfonyl)phenyl]thiazole; 5-(4-fluorophenyl)-4-(4-methylsulfonylphenyl)-2-trifluoromethylthiazole; 1-methylsulfonyl-4-[1,1-dimethyl-4-(4-fluorophenyl)cyclopenta-2,4-dien-3-yl]benzene; 4-[4-(4-fluorophenyl)-1,1-dimethylcyclopenta-2,4-dien-3-yl]benzenesulfonamide; 5-(4-fluorophenyl)-6-[4-(methylsulfonyl)phenyl]spiro[2.4]hepta-4,6-diene; 4-[6-(4-fluorophenyl)spiro[2.4]hepta-4,6-dien-5-yl]benzenesulfonamide; 6-(4-fluorophenyl)-2-methoxy-5-[4-(methylsulfonyl)phenyl]-pyridine-3-carbonitrile; 2-bromo-6-(4-fluorophenyl)-5-[4-(methylsulfonyl)phenyl]-pyridine-3-carbon itrile; 6-(4-fluorophenyl)-5-[4-(methylsulfonyl)phenyl]-2-phenyl-pyridine-3-carbonitrile; 4-[2-(4-methylpyridin-2-yl)-4-(trifluoromethyl)-1H-imidazol-1-yl]benzenesulfonamide; 4-[2-(5-methylpyridin-3-yl)-4-(trifluoromethyl)-1H-imidazol-1-yl]benzenesulfonamide; 4-[2-(2-methylpyridin-3-yl)-4-(trifluoromethyl)-1H-imidazol-1-yl]benzenesulfonamide; 3-[1-[4-(methylsulfonyl)phenyl]-4-(trifluoromethyl)-1H-imidazol-2-yl]pyridine; 2-[1-[4-(methylsulfonyl)phenyl-4-(trifluoromethyl)-1H-imidazol-2-yl]pyridine; 2-methyl-4-[1-[4-(methylsulfonyl)phenyl-4-(trifluoromethyl)-1H-imidazol-2-yl]pyridine; 2-methyl-6-[1-[4-(methylsulfonyl)phenyl-4-(trifluoromethyl)-1H-imidazol-2-yl]pyridine; 4-[2-(6-methylpyridin-3-yl)-4-(trifluoromethyl)-1H-imidazol-1-yl]benzenesulfonamide; 2-(3,4-difluorophenyl)-1-[4-(methylsulfonyl)phenyl]-4-(trifluoromethyl)-1H-imidazole; 4-[2-(4-methylphenyl)-4-(trifluoromethyl)-1H-imidazol-1-yl]benzenesulfonamide; 2-(4-chlorophenyl)-1-[4-(methylsulfonyl)phenyl]-4-methyl-1H-imidazole; 2-(4-chlorophenyl)-1-[4-(methylsulfonyl)phenyl]-4-phenyl-1H-imidazole; 2-(4-chlorophenyl)-4-(4-fluorophenyl)-1-[4-(methylsulfonyl)phenyl]-1H-imidazole; 2-(3-fluoro-4-methoxyphenyl)-1-[4-(methylsulfonyl)phenyl-4-(trifluoromethyl)-1H-imidazole; 1-[4-(methylsulfonyl)phenyl]-2-phenyl-4-trifluoromethyl-1H-imidazole; 2-(4-methylphenyl)-1-[4-(methylsulfonyl)phenyl]-4-trifluoromethyl-1H-imidazole; 4-[2-(3-chloro-4-methylphenyl)-4-(trifluoromethyl)-1H-imidazol-1-yl]benzenesulfonamide; 2-(3-fluoro-5-methylphenyl)-1-[4-(methylsulfonyl)phenyl]-4-(trifluoromethyl)-1H-imidazole; 4-[2-(3-fluoro-5-methylphenyl)-4-(trifluoromethyl)-1H-imidazol-1-yl]benzenesulfonamide; 2-(3-methylphenyl)-1-[4-(methylsulfonyl)phenyl]-4-trifluoromethyl-1H-imidazole; 4-[2-(3-methylphenyl)-4-trifluoromethyl-1H-imidazol-1-yl]benzenesulfonamide; 1-[4-(methylsulfonyl)phenyl]-2-(3-chlorophenyl)-4-trifluoromethyl-1H-imidazole; 4-[2-(3-chlorophenyl)-4-trifluoromethyl-1H-imidazol-1-yl]benzenesulfonamide; 4-[2-phenyl-4-trifluoromethyl-1H-imidazol-1-yl]benzenesulfonamide; 4-[2-(4-methoxy-3-chlorophenyl)-4-trifluoromethyl-1H-imidazol-1-yl]benzenesulfonamide; 1-allyl-4-(4-fluorophenyl)-3-[4-(methylsulfonyl)phenyl]-5-(trifluoromethyl)-1H-pyrazole; 4-[1-ethyl-4-(4-fluorophenyl)-5-(trifluoromethyl)-1H-pyrazol-3-yl]benzenesulfonamide; N-phenyl-[4-(4-fluorophenyl)-3-[4-(methylsulfonyl)phenyl]-5-(trifluoromethyl)-1H-pyrazol-1-yl]acetamide; ethyl [4-(4-fluorophenyl)-3-[4-(methylsulfonyl)phenyl]-5-(trifluoromethyl)-1H-pyrazol-1-yl]acetate; 4-(4-fluorophenyl)-3-[4-(methylsulfonyl)phenyl]-1-(2-phenylethyl)-1H-pyrazole; 4-(4-fluorophenyl)-3-[4-(methylsulfonyl)phenyl]-1-(2-phenylethyl)-5-(trifluoromethyl)pyrazole; 1-ethyl-4-(4-fluorophenyl)-3-[4-(methylsulfonyl)phenyl]-5-(trifluoromethyl)-1H-pyrazole; 5-(4-fluorophenyl)-4-(4-methylsulfonylphenyl)-2-trifluoromethyl-1H-imidazole; 4-[4-(methylsulfonyl)phenyl]-5-(2-thiophenyl)-2-(trifluoromethyl)-1H-imidazole; 5-(4-fluorophenyl)-2-methoxy-4-[4-(methylsulfonyl)phenyl]-6-(trifluoromethyl)pyridine; 2-ethoxy-5-(4-fluorophenyl)-4-[4-(methylsulfonyl)phenyl]-6-(trifluoromethyl)pyridine; 5-(4-fluorophenyl)-4-[4-(methylsulfonyl)phenyl]-2-(2-propynyloxy)-6-(trifluoromethyl)pyridine; 2-bromo-5-(4-fluorophenyl)-4-[4-(methylsulfonyl)phenyl]-6-(trifluoromethyl)pyridine; 4-[2-(3-chloro-4-methoxyphenyl)-4,5-difluorophenyl]benzenesulfonamide; 1-(4-fluorophenyl)-2-[4-(methylsulfonyl)phenyl]benzene; 5-difluoromethyl-4-(4-methylsulfonylphenyl)-3-phenylisoxazole; 4-[3-ethyl-5-phenylisoxazol-4-yl]benzenesulfonamide; 4-[5-difluoromethyl-3-phenylisoxazol-4-yl]benzenesulfonamide; 4-[5-hydroxymethyl-3-phenylisoxazol-4-yl]benzenesulfonamide; 4-[5-methyl-3-phenyl-isoxazol-4-yl]benzenesulfonamide; 1-[2-(4-fluorophenyl)cyclopenten-1-yl]-4-(methylsulfonyl)benzene; 1-[2-(4-fluoro-2-methylphenyl)cyclopenten-1-yl]-4-(methylsulfonyl)benzene; 1-[2-(4-chlorophenyl)cyclopenten-1-yl]-4-(methylsulfonyl)benzene; 1-[2-(2,4-dichlorophenyl)cyclopenten-1-yl]-4-(methylsulfonyl)benzene; 1-[2-(4-trifluoromethylphenyl)cyclopenten-1-yl]-4-(methylsulfonyl)benzene; 1-[2-(4-methylthiophenyl)cyclopenten-1-yl]-4-(methylsulfonyl)benzene; 1-[2-(4-fluorophenyl)-4,4-dimethylcyclopenten-1-yl]-4-(methylsulfonyl)benzene; 4-[2-(4-fluorophenyl)-4,4-dimethylcyclopenten-1-yl]benzenesulfonamide; 1-[2-(4-chlorophenyl)-4,4-dimethylcyclopenten-1-yl]-4-(methylsulfonyl)benzene; 4-[2-(4-chlorophenyl)-4,4-dimethylcyclopenten-1-yl]benzenesulfonamide; 4-[2-(4-fluorophenyl)cyclopenten-1-yl]benzenesulfonamide; 4-[2-(4-chlorophenyl)cyclopenten-1-yl]benzenesulfonamide; 1-[2-(4-methoxyphenyl)cyclopenten-1-yl]-4-(methylsulfonyl)benzene; 1-[2-(2,3-difluorophenyl)cyclopenten-1-yl]-4-(methylsulfonyl)benzene; 4-[2-(3-fluoro-4-methoxyphenyl)cyclopenten-1-yl]benzenesulfonamide; 1-[2-(3-chloro-4-methoxyphenyl)cyclopenten-1-yl]-4-(methylsulfonyl)benzene; 4-[2-(3-chloro-4-fluorophenyl)cyclopenten-1-yl]benzenesulfonamide; 4-[2-(2-methylpyridin-5-yl)cyclopenten-1-yl]benzenesulfonamide; ethyl 2-[4-(4-fluorophenyl)-5-[4-(methylsulfonyl)phenyl]oxazol-2-yl]-2-benzyl-acetate; 2-[4-(4-fluorophenyl)-5-[4-(methylsulfonyl)phenyl]oxazol-2-yl]acetic acid; 2-(tert-butyl)-4-(4-fluorophenyl)-5-[4-(methylsulfonyl)phenyl]oxazole; 4-(4-fluorophenyl)-5-[4-(methylsulfonyl)phenyl]-2-phenyloxazole; 4-(4-fluorophenyl)-2-methyl-5-[4-(methylsulfonyl)phenyl]oxazole; 4-[5-(3-fluoro-4-methoxyphenyl)-2-trifluoromethyl-4-oxazolyl]benzenesulfonamide; salts thereof, isomers thereof, and/or mixtures thereof.

Cox-2 inhibitors that are useful in the methods and compositions of present invention can be supplied by any source as long as the Cox-2 inhibitor is pharmaceutically acceptable. Likewise, Cox-2 inhibitors that are useful in the compositions and methods of present invention can be synthesized, for example, according to the description in Example 1. Several Cox-2 inhibitors that are suitable for use with the compositions and methods of the present invention may be synthesized by the methods described in, for example, in U.S. Pat. No. 5,466,823 to Talley, et al.

Various classes of Cox-2 inhibitors useful in the present invention can be prepared as follows. Pyrazoles can be prepared by methods described in WO 95/15316. Pyrazoles can further be prepared by methods described in WO 95/15315. Pyrazoles can also be prepared by methods described in WO 96/03385. Thiophene analogs useful in the present invention can be prepared by methods described in WO 95/00501. Preparation of thiophene analogs is also described in WO 94/15932. Oxazoles useful in the present invention can be prepared by the methods described in WO 95/00501. Preparation of oxazoles is also described in WO 94/27980. Isoxazoles useful in the present invention can be prepared by the methods described in WO 96/25405. Imidazoles useful in the present invention can be prepared by the methods described in WO 96/03388. Preparation of imidazoles is also described in WO 96/03387. Cyclopentene Cox-2 inhibitors useful in the present invention can be prepared by the methods described in U.S. Pat. No. 5,344,991. Preparation of cyclopentene Cox-2 inhibitors is also described in WO 95/00501. Terphenyl compounds useful in the present invention can be prepared by the methods described in WO 96/16934. Thiazole compounds useful in the present invention can be prepared by the methods described in WO 96/03,392. Pyridine compounds useful in the present invention can be prepared by the methods described in WO 96/03392. The preparation of pyridine compounds is also described in WO 96/24,585. Benzopyranopyrazolyl compounds useful in the present invention can be prepared by the methods described in WO 96/09304. Chromene compounds useful in the present invention can be prepared by the methods described in WO 98/47890. Preparation of chromene compounds is also described in WO 00/23433. Chromene compounds can further be prepared by the methods described in U.S. Pat. No. 6,077,850. Preparation of chromene compounds is further described in U.S. Pat. No. 6,034,256. Arylpyridazinones useful in the present invention can be prepared by the methods described in WO 00/24719. Preparation of arylpyridazinones is also described in WO 99/10332. Arylpyridazinones can further be prepared by the methods described in WO 99/10331. 5-Alkyl-2-arylaminophenylacetic acids and derivatives useful in the present invention can be prepared by the methods described in WO 99/11605. Diarylmethylidenefuran derivative Cox-2 selective inhibitors useful in the present invention can be prepared by the methods described in U.S. Pat. No. 6,180,651. The celecoxib used in the compositions and methods of the present invention can be prepared in the manner set forth in U.S. Pat. No. 5,466,823. The valdecoxib used in the compositions and methods of the present invention can be prepared in the manner set forth in U.S. Pat. No. 5,633,272. The parecoxib used in the compositions and methods of the present invention can be prepared in the manner set forth in U.S. Pat. No. 5,932,598. The rofecoxib used in the compositions and methods of the present invention can be prepared in the manner set forth in U.S. Pat. No. 5,474,995. The deracoxib used in the compositions and methods of the present invention can be prepared in the manner set forth in U.S. Pat. No. 5,521,207. The etoricoxib used in the compositions and methods of the present invention can be prepared in the manner set forth in WO 98/03484. The cimicoxib used in the compositions and methods of the present invention can be prepared in the manner set forth in Drugs of the Future, 29(4):325-330 (2004). The meloxicam used in the compositions and methods of the present invention can be prepared in the manner set forth in U.S. Pat. No. 4,233,299. The compound 4-(4-cyclohexyl-2-methyloxazol-5-yl)-2-fluorobenzenesulfonamide used in the compositions and methods of the present invention can be prepared in the manner set forth in U.S. Pat. No. 5,994,381. The compound 2-(3,4-difluorophenyl)-4-(3-hydroxy-3-methylbutoxy)-5-[4-(methylsulfonyl)phenyl]-3(2H)-pyridazinone used in the compositions and methods of the present invention can be prepared in the manner set forth in WO 00/24719. The compound 2-(3,5-difluorophenyl)-3-[4-(methylsulfonyl)phenyl]-2-cyclopenten-1-one used in the compositions and methods of the present invention can be prepared in the manner set forth in EP 863134. The compound 2-[(2-chloro-6-fluorophenyl)amino]-5-methyl-benzeneacetic acid used in the compositions and methods of the present invention can be prepared in the manner set forth in WO 99/11605. The compound N-[2-(cyclohexyloxy)-4-nitrophenyl]methanesulfonamide used in the compositions and methods of the present invention can be prepared in the manner set forth in U.S. Pat. No. 4,885,367. The compound (3Z)-3-[(4-chlorophenyl)[4-(methylsulfonyl)phenyl]methylene]dihydro-2(3H)-furanone used in the compositions and methods of the present invention can be prepared in the manner set forth in U.S. Pat. No. 6,180,651.

Cox-2 inhibitors can also be isolated and purified from natural sources. Cox-2 inhibitors should be of a quality and purity that is conventional in the trade for use in pharmaceutical products.

Preferred Cox-2 selective inhibitor compounds are those compounds selected from the group consisting of celecoxib, parecoxib, deracoxib, valdecoxib, etoricoxib, meloxicam, tilmacoxib, cimicoxib, rofecoxib, lumiracoxib, etoricoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, SD-8381, prodrugs of any of them, and mixtures thereof.

More preferred is that the cyclooxygenase-2 selective inhibitor is selected from the group consisting of celecoxib, parecoxib, deracoxib, valdecoxib, lumiracoxib, etoricoxib, rofecoxib, prodrugs of any of them, and mixtures thereof.

Even more preferred still is that the cyclooxygenase-2 selective inhibitor comprises celecoxib.

In one embodiment of the present invention, the Cox-2 selective inhibitor is administered from about 0.1 mg per kg to about 25 mg per kg subject body weight.

In one embodiment of the present invention, the Cox-2 selective inhibitor is administered from about 0.5 mg per kg to about 10 mg per kg subject body weight.

The amount of the intraocular pressure reducing agent that is used in the subject method may be an amount that, when administered with the Cox-2 inhibitor, is sufficient to constitute an effective amount of the combination. Preferably, such amount would be sufficient to provide a therapeutically effective amount of the combination.

In the present method, the amount of the intraocular pressure reducing agent that is used in the novel method of treatment preferably ranges from about 0.001 to about 500 micrograms per day per kilogram of body weight of the subject (μg/day·kg), more preferably from about 0.01 to about 50 μg/day kg, even more preferably from about 0.02 to about 10 μg/day·kg, and yet more preferably from about 0.03 to about 8 μg/day·kg.

For topical administration, such as in eye drops, when the IOP reducing agent is unoprostone isopropyl, the preferred dosage amount is about 5 μg/day·kg; when the IOP reducing agent is travoprost, the preferred dosage amount is about 0.035 μg/day·kg; and when the IOP reducing agent is latanoprost, the preferred dosage amount is about 0.043 μg/day·kg. Expressed differently, the IOP reducing agent can be present in eye drop formulations in a concentration of from about 0.1% to about 20% by weight, and the drops can be administered to each eye of a subject at the rate of from about one 40 microliter drop every week to about 4 such drops per day. Preferably, eye drops having from about 0.1% to about 20% by weight of the IOP reducing agent are administered to each eye of the subject at the rate of one 40 microliter drop per day.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages. For purposes of calculating dosages, it is assumed that the weight of a normal human adult subject is 70 kilograms.

One embodiment of the present invention comprises a method for the prevention or treatment of an optic neuropathy in a subject in need of such prevention or treatment. The method comprises administering to the subject a Cox-2 inhibitor and an intraocular pressure reducing agent.

As used herein, the terms “optic neuropathy”, or “optic neuropathies” are intended to include diseases, disorders, or damage to the nerves or other structures of the eye. By way of example, such optic neuropathies include uveitis, such as anterior uveitis, intermediate uveitis, posterior uveitis, and diffuse uveitis; uveitic syndromes, such as ankylosing spondylitis, juvenile rheumatoid arthritis, Behçet's syndrome, pars planitis, toxoplasmosis, cytomegalovirus, inflammation caused by herpes zoster, inflammation caused by herpes simplex, toxocariasis, birdshot chorioretinopathy, presumed ocular histoplasmosis syndrome, syphilis, tuberculosis, Vogt-Koyanagi-Harada syndrome, sympathetic ophthalmia, ocular sarcoidosis and endophthalmitis; masquerade syndromes, such as intraocular malignancy, retinitis pigmentosa, and reactions to drugs; vascular retinopathies, such as hypertensive retinopathy, diabetic retinopathy, central retinal artery occlusion, and central retinal vein occlusion; age-related macular degeneration; retinitis pigmentosa; glaucoma; ocular hypertension; optic nerve and pathway disorders, such as papilledema, papillitis, retrobulbar neuritis, toxic amblyopia, optic atrophy, bitemporal hemianopia, and homonymous hemianopia.

The present method is preferred for use in the prevention and/or treatment of glaucoma. In the present application, the term “glaucoma” is intended to include chronic (idiopathic) open-angle glaucomas, pupillary block glaucomas, developmental glaucomas, glaucomas associated with other ocular disorders, glaucomas associated with elevated episcleral venous pressure, glaucomas associated with inflammation and trauma, and glaucomas following intraocular surgery.

Examples of chronic (idiopathic) open-angle glaucomas include high-pressure glaucomas and normal-pressure glaucomas. Examples of pupillary block glaucomas include acute angle-closure glaucoma, subacute angle-closure glaucoma, chronic angle-closure glaucoma, and combined mechanism glaucoma. Examples of developmental glaucomas include congenital (infantile) glaucoma, juvenile glaucoma, Axenfeld-Rieger syndrome, Peter's anomaly, aniridia and other developmental anomalies. Examples of glaucomas associated with other ocular disorders include glaucomas associated with disorders of the corneal endothelium, such as iridocorneal endothelial syndrome, posterior polymorphous dystrophy, and Fuch's endothelial dystrophy; glaucomas associated with disorders of the iris and ciliary body, such as pigmentary glaucoma, iridoschisis, and plateau iris; glaucomas associated with disorders of the lens, such as exfoliation syndromes, lens-induced open-angle glaucomas, and glaucomas associated with lens intumescence and dislocation; glaucomas associated with disorders of the retina, choroid, and vitreous, including glaucomas associated with retinal detachment and vitreoretinal abnormalities; and neovascular glaucomas. Examples of glaucomas associated with elevated episcleral venous pressure include systemic diseases with associated elevated intraocular pressure and glaucoma, and corticosteroid-induced glaucoma. Examples of glaucomas associated with inflammation and trauma include glaucomas associated with keratitis, episcleritis, and scleritis; glaucomas associated with uveitis; glaucomas associated with ocular trauma; and glaucomas associated with hemorrhage. Examples of glaucomas following intraocular surgery include ciliary block (malignant) glaucoma, glaucomas in aphakia and pseudophakia, epithelial, fibrous, and endothelial proliferation, glaucomas associated with corneal surgery, and glaucomas associated with vitreoretinal surgery.

The combination of a Cox-2 inhibitor and an intraocular pressure reducing agent can be supplied in the form of a novel therapeutic composition that is believed to be within the scope of the present invention.

The combination of a Cox-2 inhibitor and an intraocular pressure reducing agent can be provided in a pharmaceutically acceptable carrier or excipient to form a pharmaceutical composition.

The terms “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in the combination of the invention are the isomeric forms and tautomers and the pharmaceutically-acceptable salts of Cox-2 inhibitors and intraocular pressure reducing agents. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, β-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

Examples of pharmaceutically acceptable carriers or excipients include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not negated or inhibited to such an extent that treatment is ineffective. In one embodiment, the Cox-2 inhibitor and the intraocular pressure reducing agent are administered to a subject together in one pharmaceutical carrier. In another embodiment, they are administered separately.

The pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

In particular, the combinations of the present invention can be administered topically into the eye in the form of liquid drops. Eye drops can be formulated to contain a suitable amout of the active agents along with various formulatory ingredients, such as antimicrobial preservatives and tonicity agents. Examples of suitable antimicrobial preservatives include: benzalkonium chloride, thimerosal, chlorobutanol, methyl paraben, propyl paraben, phenylethyl alcohol, edetate disodium, sorbic acid, and other agents equally well-known to those skilled in the art. Such preservatives, if utilized, will typically be employed in an amount between about 0.001% and about 1.0% by weight. Examples of suitable agents which may be used to adjust the tonicity or osmolality of the formulations include: sodium chloride, potassium chloride, mannitol, dextrose, glycerin, and propylene glycol. Such agents, if utilized, will typically be employed in an amount between about 0.1% and about 10.0% by weight.

The present therapeutic and pharmaceutical compositions can be formulated in various dosage forms suitable for topical ophthalmic delivery, including solutions, suspensions, emulsions, gels and erodible solid ocular inserts.

The subject method of administering a Cox-2 inhibitor alone or in combination with an intraocular pressure reducing agent and compositions comprising the same can also be administered parenterally, either subcutaneously, or intravenously, or intramuscularly, or intrasternally, or by infusion techniques, in the form of sterile injectable aqueous or oleaginous suspensions.

Aqueous suspensions can be produced that contain the active materials in a mixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

Oral (intra-gastric) is another preferred route of administration for the combination therapy. Pharmaceutically acceptable carriers can be in solid dosage forms for the methods of the present invention, which include tablets, capsules, pills, and granules, which can be prepared with coatings and shells, such as enteric coatings and others well known in the art. Liquid dosage forms for oral administration include pharmaceutically acceptable emulsions, solutions, suspensions, syrups, and elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the Cox-2 inhibitor and the intraocular pressure reducing agent may be formulated with sweetening agents, for example glycerol, sorbitol, or sucrose. Such formulations may also contain a demulcent, a preservative and flavoring and coloring agents.

Administration can also be by inhalation, in the form of aerosols or solutions for nebulizers, or rectally, in the form of suppositories prepared by mixing the drug with a suitable non-irritating excipient which is solid at ordinary temperature, but liquid at the rectal temperature and will therefore, melt in the rectum to release the drug. Such materials are cocoa butter and polyethylene glycols.

Also encompassed by the present invention is buccal or “sub-lingual” administration, which includes lozenges or a chewable gum comprising the compounds, set forth herein. The compounds can be deposited in a flavored base, usually sucrose, and acacia or tragacanth, and pastilles comprising the compounds in an inert base such as gelatin and glycerin or sucrose and acacia.

Other methods for administration of the Cox-2 inhibitor compound and the intraocular pressure reducing agent include dermal patches that release the medicaments directly into a subject's skin.

Topical delivery systems are also encompassed by the present invention and include ointments, powders, sprays, creams, jellies, collyriums, solutions or suspensions.

The solubility of the components of the present compositions may be enhanced by a surfactant or other appropriate co-solvent in the composition. Such co-solvents include polysorbate 20, 60, and 80, polyoxyethylene/polyoxypropylene surfactants (e.g. Pluronic F-68, F-84 and P-103), cyclodextrin, or other agents known to those skilled in the art. Typically, such co-solvents are employed at a level of from 0.01% to 2% by weight.

A penetration enhancer is an agent used to increase the permeability of the skin to an active agent to increase the rate at which the drug diffuses through the skin and enters the tissues and bloodstream. Thus, in one embodiment of the present invention, a penetration enhancer may be added to a Cox-2 inhibitor and intraocular pressure reducing agent topical composition.

Examples of penetration enhancers suitable for use with the compositions of the present invention include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof.

The above considerations concerning effective formulations and administration procedures are well known in the art and are described in standard textbooks. See e.g. Gennaro, A. R., Remington: The Science and Practice of Pharmacy, 20^{th} Edition, (Lippincott, Williams and Wilkins), 2000; Hoover, John E., Remington's Pharmaceutical Sciences, Mack Publishing Co., Easton Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

The present invention further comprises kits that are suitable for use in performing the methods of treatment described above. In one embodiment, the kit contains a first dosage form comprising a Cox-2 inhibitor in one or more of the forms identified above and a second dosage form comprising an intraocular pressure reducing agent, in amounts which comprise a therapeutically effective combination for the prevention or treatment of an optic neuropathy. Preferably, the first dosage form and the second dosage form together comprise a therapeutically effective amount of the compounds for the prevention or treatment of an optic neuropathy.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### EXAMPLE 1

This example shows the preparation of celecoxib.

### Step 1: Preparation of 1-(4-methylphenyl)-4,4,4-trifluorobutane-1,3-dione

Following the disclosure provided in U.S. Pat. No. 5,760,068, 4′-Methylacetophenone (5.26 g, 39.2 mmol) was dissolved in 25 mL of methanol under argon and 12 mL (52.5 mmol) sodium methoxide in methanol (25%) was added. The mixture was stirred for 5 minutes and 5.5 mL (46.2 mmol) ethyl trifluoroacetate was added. After refluxing for 24 hours, the mixture was cooled to room temperature and concentrated. 100 mL 10% HCl was added and the mixture extracted with 4×75 mL ethyl acetate. The extracts were dried over MgSO₄, filtered and concentrated to afford 8.47 g (94%) of a brown oil which was carried on without further purification.

### Step 2: Preparation of 4-[5-(4-methylphenyl)-3-(trifluoromethyl)-1H-pyrazol-1-yl]benzenesulfonamide

To the dione from Step 1 (4.14 g, 18.0 mmol) in 75 mL absolute ethanol, 4.26 g (19.0 mmol) 4-sulphonamidophenylhydrazine hydrochloride was added. The reaction was refluxed under argon for 24 hours. After cooling to room temperature and filtering, the reaction mixture was concentrated to afford 6.13 g of an orange solid. The solid was recrystallized from methylene chloride/hexane to give 3.11 g (8.2 mmol, 46%) of the product as a pale yellow solid, having a melting point (mp) of 157°-159° C.; and a calculated composition of C₁₇H₁₄N₃O₂SF₃; C, 53.54; H, 3.70; N, 11.02. The composition that was found by analysis was: C, 53.17; H, 3.81; N, 10.90.

### EXAMPLE 2

This example shows the preparation of ophthalmic solution containing travoprost and celecoxib.

Celecoxib can be prepared as described in Example 1 or, alternatively, can be obtained under the trade name CELEBREX® from Pharmacia Corporation, Peapack, N.J.

Travoprost is a synthetic prostaglandin F2α analogue, its chemical name is isopropyl(Z)-7-[(1R,2R,3R,5S)-3,5-dihydroxy-[(1E,3R)-3-hydroxy-4-[(α,α,α,-trifluoro-m-tolyl)oxy]-1-butenyl]cyclopentyl]-5-heptenoate. Travoprost can be obtained from Alcon Laboratories, Inc., Fort Worth, Tex., under the trade name TRAVATAN®.

An ophthalmic solution can be prepared by intermixing celecoxib (10 g) and travoprost (0.04 g) into solution in sterile water (1 liter) with 0.02% benzalkonium chloride, and with sodium chloride, sodium dihydrogen phosphate monohydrate, and disodium hydrogen phosphate anhydrous at levels suitable for providing an isotonic solution buffered at a pH of about 6.7 and an osmolality of about 265 mOsmol/kg. After all materials are in solution, the solution is ready for use or storage. Normal dosage for a human is one drop per eye per day.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense. In addition, it should be understood that aspects of the various embodiments may be interchanged both in whole or in part.

1. A method for the prevention or treatment of an optic neuropathy, the treatment comprising administering to the subject a cyclooxygenase-2 inhibitor and an intraocular pressure reducing agent.

2. The method according to claim 1, wherein the amount of the cyclooxygenase-2 inhibitor and the amount of the intraocular pressure reducing agent are such that the amount of the combination is effective for the prevention or treatment of the optic neuropathy.

3. The method according to claim 1, wherein the intraocular pressure reducing agent comprises at least one compound that is selected from the group consisting of direct-acting miotics, cholinergic agonists, indirect-acting miotics, cholinesterase inhibitors, carbonic anhydrase inhibitors, nonselective adrenergic agonists, α₂-selective adrenergic agonists, β-blockers, prostaglandin analogues, osmotic diuretics, p38 kinase antagonists, salts thereof, isomers thereof, prodrugs thereof, and mixtures of any of these.

4. The method according to claim 1, wherein the intraocular pressure reducing agent comprises at least one compound that is selected from the group consisting of pilocarpine, carbachol, acetylcholinesterase inhibitors, physostigmine, neostigmine, demecarium, echothiophate iodide, isoflurophate, acetazolamide, dichlorphenamide, methazolamide, ethoxzolamide, dorzolamide, epinephrine, dipivalylepinephrine, dipivefrin, aprachlonidine, brimonidine, timolol, betaxolol, levobunolol, carteolol, metipranolol, F series prostaglandin analogues, E series prostaglandin analogues, D series prostaglandin analogues, glycerin, mannitol, isosorbide, salts thereof, isomers thereof, prodrugs thereof, and mixtures of any of these.

5. The method according to claim 1, wherein the intraocular pressure reducing agent comprises a prostaglandin analogue.

6. The method according to claim 5, wherein the prostaglandin analogue comprises a prostaglandin F_{2α} analogue.

7. The method according to claim 1, wherein the intraocular pressure reducing agent comprises an agent selected from latanoprost, travoprost, AL-5848, PhXA85, unoprostone, bimatoprost, and pharmaceutically acceptable salts and prodrugs thereof.

8. The method according to claim 7, wherein the intraocular pressure reducing agent comprises latanoprost and pharmaceutically acceptable salts and prodrugs thereof.

9. The method according to claim 7, wherein the intraocular pressure reducing agent comprises travoprost and pharmaceutically acceptable salts and prodrugs thereof.

10. The method according to claim 7, wherein the intraocular pressure reducing agent comprises AL-5848 and pharmaceutically acceptable salts and prodrugs thereof.

11. The method according to claim 7, wherein the intraocular pressure reducing agent comprises unoprostone and pharmaceutically acceptable salts and prodrugs thereof.

12. The method according to claim 7, wherein the intraocular pressure reducing agent comprises PhXA85 and pharmaceutically acceptable salts and prodrugs thereof.

13. The method according to claim 7, wherein the intraocular pressure reducing agent comprises bimatoprost and pharmaceutically acceptable salts and prodrugs thereof.

14. The method according to claim 1, wherein the cyclooxygenase-2 inhibitor comprises a non-steroidal anti-inflammatory drug.

15. The method according to claim 14, wherein the cyclooxygenase-2 inhibitor comprises at least one compound that is selected from the group consisting of ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam 2-fluoro-a-methyl[1,1′-biphenyl]-4-acetic acid, 4-(nitrooxy)butyl ester, salts thereof, isomers thereof, prodrugs thereof, and mixtures of any of these.

16. The method according to claim 1, wherein the cyclooxygenase-2 inhibitor comprises a cyclooxygenase-2 selective inhibitor.

17. The method according to claim 16, wherein the cyclooxygenase-2 selective inhibitor comprises at least one compound that is selected from the group consisting of celecoxib, parecoxib, deracoxib, valdecoxib, etoricoxib, meloxicam, tilmacoxib, cimicoxib, rofecoxib, lumiracoxib, etoricoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, SD-8381, a chromene Cox-2 inhibitor, salts thereof, isomers thereof, prodrugs thereof, and mixtures of any of these.

18. The method according to claim 16, wherein the cyclooxygenase-2 selective inhibitor comprises at least one compound that is selected from the group consisting of celecoxib, parecoxib, deracoxib, valdecoxib, etoricoxib, meloxicam, rofecoxib, lumiracoxib, a chromene Cox-2 inhibitor, salts thereof, isomers thereof, prodrugs thereof, and mixtures of any of these.

19. The method according to claim 1, wherein the cycloxygenase-2 inhibitor comprises at least one compound that is selected from the group consisting of a chromene Cox-2 selective inhibitor, lumiricoxib, RS 57067, NS-398, BMS 347070, ABT-963, SD-8381, PAC-10549, PAC-10649, salts thereof, isomers thereof, prodrugs thereof, and mixtures of any of these.

20. The method according to claim 8, wherein the cyclooxygenase-2 selective inhibitor comprises celecoxib.

21. The method according to claim 1, wherein the optic neuropathy is selected from the group consisting of uveitis, uveitic syndromes, masquerade syndromes, vascular retinopathies, age-related macular degeneration, retinitis pigmentosa, glaucoma, ocular hypertension, optic nerve and pathway disorders.

22. The method according to claim 21, wherein the uveitis is selected from the group consisting of anterior uveitis, intermediate uveitis, posterior uveitis, and diffuse uveitis.

23. The method according to claim 21, wherein the uveitic syndrome is selected from the group consisting of ankylosing spondylitis, juvenile rheumatoid arthritis, Behçet's syndrome, pars planitis, toxoplasmosis, cytomegalovirus, inflammation caused by herpes zoster, inflammation caused by herpes simplex, toxocariasis, birdshot chorioretinopathy, presumed ocular histoplasmosis syndrome, syphilis, tuberculosis, Vogt-Koyanagi-Harada syndrome, sympathetic ophthalmia, ocular sarcoidosis and endophthalmitis.

24. The method according to claim 21, wherein the masquerade syndrome is selected from the group consisting of intraocular malignancy, retinitis pigmentosa, and reactions to drugs.

25. The method according to claim 21, wherein the vascular retinopathy is selected from the group consisting of hypertensive retinopathy, diabetic retinopathy, central retinal artery occlusion, and central retinal vein occlusion.

26. The method according to claim 21, wherein the optic nerve and pathway disorder is selected from the group consisting of papilledema, papillitis, retrobulbar neuritis, toxic amblyopia, optic atrophy, bitemporal hemianopia, and homonymous hemianopia.

27. The method according to claim 21, wherein the glaucoma is selected from the group consisting of chronic (idiopathic) open-angle glaucomas, pupillary block glaucomas, developmental glaucomas, glaucomas associated with other ocular disorders, glaucomas associated with elevated episcleral venous pressure, glaucomas associated with inflammation and trauma, and glaucomas following intraocular surgery.

28. The method according to claim 27, wherein the chronic (idiopathic) open-angle glaucoma is selected from the group consisting of high-pressure glaucomas and normal-pressure glaucomas.

29. The method according to claim 27, wherein the pupillary block glaucoma is selected from the group consisting of acute angle-closure glaucoma, subacute angle-closure glaucoma, chronic angle-closure glaucoma, and combined mechanism glaucoma.

30. The method according to claim 27, wherein the developmental glaucoma is selected from the group consisting of congenital (infantile) glaucoma, juvenile glaucoma, Axenfeld-Rieger syndrome, Peter's anomaly, aniridia and other developmental anomalies.

31. The method according to claim 27, wherein the glaucoma associated with other ocular disorders is selected from the group consisting of glaucomas associated with disorders of the corneal endothelium, glaucomas associated with disorders of the iris and ciliary body, glaucomas associated with disorders of the lens, glaucomas associated with disorders of the retina, choroid, and vitreous, glaucomas associated with retinal detachment and vitreoretinal abnormalities; and neovascular glaucomas.

32. The method according to claim 31, wherein the glaucomas associated with disorders of the corneal endothelium is selected from the group consisting of iridocorneal endothelial syndrome, posterior polymorphous dystrophy, and Fuch's endothelial dystrophy.

33. The method according to claim 31, wherein the glaucoma associated with disorders of the iris and ciliary body is selected from the group consisting of pigmentary glaucoma, iridoschisis, and plateau iris.

34. The method according to claim 31, wherein the glaucoma associated with disorders of the lens is selected from the group consisting of exfoliation syndromes, lens-induced open-angle glaucomas, and glaucomas associated with lens intumescence and dislocation.

35. The method according to claim 27, wherein the glaucoma associated with inflammation and trauma is selected from the group consisting of glaucomas associated with keratitis, episcleritis, and scleritis.

36. The method according to claim 27, wherein the glaucoma following intraocular surgery is selected from the group consisting of ciliary block (malignant) glaucoma, glaucomas in aphakia and pseudophakia, epithelial, fibrous, and endothelial proliferation, glaucomas associated with corneal surgery, and glaucomas associated with vitreoretinal surgery

37. The method according to claim 1, wherein the subject is an animal.

38. The method according to claim 1, wherein the subject is a human.

39. The method according to claim 1, wherein the treating step comprises administering a cyclooxygenase-2 inhibitor and an intraocular pressure reducing agent to the subject in one or more dose per day.

40. The method according to claim 39, wherein the cyclooxygenase-2 inhibitor and the intraocular pressure reducing agent are administered to the subject substantially simultaneously.

41. The method according to claim 39, wherein the cyclooxygenase-2 inhibitor and the intraocular pressure reducing agent are administered sequentially.

42. A composition comprising a Cox-2 inhibitor and an intraocular pressure reducing agent.

43. The composition according to claim 42, comprising celecoxib and travoprost.

44. The composition according to claim 42, comprising celecoxib and lantanoprost.

45. The composition according to claim 42, wherein the composition is designed for the prevention or treatment of an optic neuropathy.

46. A pharmaceutical composition comprising a pharmaceutically-acceptable excipient and a combination comprising a Cox-2 inhibitor and an intraocular pressure reducing agent.

47. A kit that is suitable for use in the prevention or treatment of an optic neuropathy, the kit comprising a first dosage form comprising a Cox-2 inhibitor and a second dosage form comprising an intraocular pressured reducing agent or prodrug thereof, in quantities which comprise a therapeutically effective amount of the combination of the compounds for the prevention or treatment of the optic neuropathy.
